(19) United States
(12) Patent Application Publication
(10) Pub. No.: US 2023/0120697 A1
(43) Pub. Date: Apr. 20, 2023

(54) SWELLABLE POLYMERIC MATERIALS AND USEFUL ARTICLES INCORPORATING SAME

(71) Applicant: SOANE MATERIALS LLC, Reno, NV (US)

(72) Inventors: David S. Soane, Coral Gables, FL (US); Allison Hope Greene, Reno, NV (US); Sydney Greenough Higgins, Miami, FL (US)

(21) Appl. No.: 17/943,538

(22) Filed: Sep. 13, 2022

Related U.S. Application Data
(60) Provisional application No. 63/245,129, filed on Sep. 16, 2021.

(51) Int. Cl. A61F 13/20; A61L 15/60; A61L 15/62; A61L 15/28; A61L 15/48

(52) U.S. Cl. CPC A61F 13/2051; A61L 15/28; A61L 15/48; A61L 15/60; A61L 15/62; A61L 2300/802

(57) ABSTRACT

The present invention provides absorbent materials comprising a hydrogel-forming swellable polymer and a plasticizer, wherein the absorbent material demonstrates an advantageous performance characteristic such as advantageous fluid absorption capacity, fluid absorption rate, and rewetting, wherein the advantageous performance characteristic is within at least about 80% of a similar characteristic exhibited by a crosslinked polyacrylate superabsorbent polymer, or wherein the cumulative performance of the advantageous performance characteristics is comparable or superior to performance exhibited by the crosslinked polyacrylate superabsorbent polymer. The present invention also relates to articles of manufacture comprising such absorbent materials, and methods of manufacture.

### RELATED APPLICATION

This application claims the benefit of U.S. Provisional Application No. 63/245,129 filed Sept. 16, 2021. The entire contents of the above application are incorporated by reference herein.

### FIELD OF THE APPLICATION

This application relates to swellable polymeric materials and articles formed therefrom.

### BACKGROUND

Superabsorbent polymers, described in more detail below, are specialized crosslinked polymeric networks characterized by the ability to absorb many times their weight in liquid while themselves remaining intact in the presence of the imbibed liquid. Their absorbent and retentive properties can make them useful in any setting in which fluids need to be absorbed and retained in a convenient form factor. Therefore, superabsorbent polymers (SAPs) form the basis of many consumer products that depend on their ability to absorb aqueous fluids, such as baby diapers, adult incontinence pads, feminine care products, and the like.

SAPs in common use are mainly derived from acrylic acid, with the polymer being formed synthetically from acrylate monomers. The acrylate monomers themselves are derived from petrochemical sources and are considered a non-renewable resource that is dependent on the petroleum industry. Moreover, the processes required to form SAPs from the acrylate monomers is energy-intensive, entailing expense and imposing environmental burdens. Presently, SAPs are estimated to make up about 27% of the weight of a conventional baby diaper, and their manufacturing contributes about 34.5% of the global warming potential (CO₂eq) impact for diaper production. The high contribution of CO₂ emissions from SAP production and processing underscores the environmental impact of conventional SAPs.

The manufacturing process for SAP hydrogels can lead to residual acrylate monomers becoming embedded in the final product. Residual monomers in SAPs can leach from the absorbent polymeric material into surrounding aqueous fluids, contacting human tissues and entering the environment. While technologies have been devised to minimize the levels of residual monomers in SAPs, the presence of these substances even in small quantities can cause skin irritation and health problems, and can contaminate the environment.

The most significant environmental hazard posed by conventional SAPs is their resistance to biodegradation. Scientific studies have demonstrated the slow rate of SAP degradation under normal environmental conditions. A study investigating the biodegradability of polyacrylate polymers used as soil conditioners found that the main long chain of polyacrylate polymers in the hydrogels degraded, “if at all, at rates of 0.12-0.24% per 6 months.” (Biodegradability of a polyacrylate superabsorbent in agricultural soil Burkhard Wilske & Mo Bai & Beate Lindenstruth & Martin Bach & Zahra Rezaie & Hans-Georg Frede & Lutz Breuer, Environ Sci Pollut Res DOI 10.1007/s11356-013-2103-1). Studies have shown that for higher molecular weight SAPs, the rate of biodegradation can be even slower.

Personal care items such as baby diapers and adult incontinence products containing SAPs are disposed of as municipal solid waste. In 2018, 3.3 million tons of disposable personal care items were relegated to landfills, according to an EPA report; this tonnage equaled about 1.4% of total municipal solid waste in the US for that year. It is estimated that a discarded disposable diaper will take approximately 450 years to decompose.

Despite the limitations of conventional SAPs as absorbent materials, including health and safety concerns and sustainability concerns, they have been adopted widely. One report indicates that 90-95% of American babies use diapers with SAPs, resulting in approximately 27.4 billion single use diapers being used every year. Disposable diapers have incorporated performance features that result in extended dryness and reduced leakage. In view of the burden that these products place on the environment, however, there is a need in the art to improve this product to be more sustainable while retaining its beneficial features. While alternatives to conventional SAPs have been proposed that are derived from natural sources, these alternatives tend not to provide the same high performance as conventional SAPs.

Therefore, an alternative to conventional polyacrylate SAPs would be advantageously derived from natural sources with less stress imposed on the environment, while providing the consumer with similar performance. Desirably, a natural and biodegradable superabsorbent polymer can provide an alternative to SAP absorbents that can be readily integrated into existing manufacturing processes for absorbent articles, thus avoiding capital expenditures and streamlining the path to commercialization. It is envisioned that such a material could be used as an absorbent in other applications, such as the management of pet waste; a biodegradable superabsorbent polymer would advantageously offer a more sustainable alternative to the clay minerals or silica gels that are used as pet waste absorbents, for example in animal litter.

### SUMMARY

Disclosed herein, in embodiments, are absorbent materials comprising at least one hydrogel-forming swellable polymer, for example, at least one bio-based hydrogel-forming swellable polymer; and a plasticizer; wherein the absorbent material demonstrates an advantageous performance characteristic selected from the group consisting of fluid absorption capacity, fluid absorption rate, and rewetting, wherein the advantageous performance characteristic is within at least about 80% of a similar characteristic exhibited by a conventional superabsorbent polymer, or wherein the cumulative performance of the advantageous performance characteristics is comparable or superior to performance exhibited by the conventional superabsorbent polymer. In embodiments, the absorbable material is biodegradable or compostable. In embodiments, the at least one polymer is a biodegradable synthetic polymer or is bio-based, and in embodiments, the at least one polymer demonstrates superabsorbent properties. In embodiments, the at least one polymer is an anionic polymer, which can be alginate or carrageenan. In embodiments, the at least one polymer is a cationic polymer or a neutral polymer. In embodiments, the polymer is a polysaccharide, which can be selected from the group consisting of dextrin, dextran, agarose, cellulose, and a derivative of any of the foregoing. In embodiments, the at least one polymer is a polysaccharide selected from the group consisting of xanthan gum, alginic acid, and sodium alginate. In embodiments, the plasticizer is selected from the group consisting of small molecules, polymeric polyols, and oligomers. In embodiments, the absorbent material comprises a second plasticizer. In embodiments, at least one of the plasticizer and the second plasticizer is a small molecule, and the small molecule is a polyol, which can be glycerol, glycerin, maltitol, or xylitol. In embodiments, the plasticizer is an oligomer, which can be a glucose oligomer or a cellulose oligomer. In embodiments, the absorbent material further comprises one or more additional bio-based hydrogel-forming swellable polymers. In embodiments, the absorbent material comprises a crosslinking agent, which can be a covalent or an ionic crosslinking agent, or a secondary crosslinking agent such as a divalent cation found in a body fluid. The absorbent material can be crosslinked in its interior, on its surface, or both. The absorbent material can further comprise a catalyst for the crosslinking agent. In embodiments, the absorbent material further comprises a plasticizer additive or a functional additive. Also disclosed herein, in embodiments, are methods of forming a solid biodegradable absorbent material in a predesignated shape, wherein the solid biodegradable absorbent material demonstrates an advantageous performance characteristic selected from the group consisting of fluid absorption capacity, fluid absorption rate, and rewetting, wherein the advantageous performance characteristic is within at least about 80% of a similar characteristic exhibited by a conventional superabsorbent polymer, or wherein the cumulative performance of the advantageous performance characteristics is comparable or superior to performance exhibited by the conventional superabsorbent polymer, wherein the method comprises preparing a liquid composition comprising at least one bio-based hydrogel-forming swellable polymer, a plasticizer, and a surfactant; processing the liquid composition in a shape-forming apparatus, wherein the shape-forming apparatus is selected from the group consisting of an extruder, a mold, an electrospinner, a slot-die, and a fluid dispenser, and wherein the shape-forming apparatus forms the liquid formulation into a selected three-dimensional configuration consistent with the predesignated shape; and solidifying the selected three-dimensional configuration, thereby producing the predesignated shape. In embodiments, the solid biodegradable absorbent material is a semi-solid material. In embodiments, the at least one bio-based hydrogel-forming polymer is a polysaccharide, the plasticizer is glycerol or xylitol, and the surfactant is capryl glucoside or hexyl glucoside. In embodiments, the predesignated shape is an elongate strand or a flattened sheet or a flat shape or an ovoid shape. In embodiments, the shape-forming apparatus is an extruder, which can have devolatilization capacity for evaporating excess water. In embodiments, the step of solidifying comprises a substep of drying the selected three-dimensional configuration to form the predesignated shape. In embodiments, the polymer is biodegradable or compostable. In embodiments, the methods further comprise a step of adding a crosslinking agent to the hydrogel mixture before the step of directing. In embodiments, the methods further comprise a step of adding an additive having advantageous properties to the hydrogel mixture before the step of directing, where the additive can be a filler additive, which can be selected from the group consisting of fluff pulp, microfibrillated cellulose, and nanofibrillated cellulose. In embodiments, the additive is an odor-absorbent additive. In embodiments, the additive has specialized properties. In embodiments, the methods further comprise a step of heating the formed absorbent material after the step of directing.

Also disclosed is an article of manufacture comprising a disposable absorbent area, wherein the disposable absorbent area comprises the absorbent material described herein, and wherein the disposable absorbent area is organized as a multilayered structure. In embodiments, the multilayered structure comprises one or more layers of absorbent material, and the absorbent material is a foamed material. In embodiments, the multilayered structure comprises at least one primary absorbent layer formed from the absorbent material and at least one secondary absorbent layer. In embodiments, the at least one primary absorbent layer can be formed as a sheet, which can be penetrated by one or more apertures. In embodiments, the at least one primary absorbent layer comprises pieces of the absorbent material overlapping each other to produce gaps that permit fluid to pass through said layer. In embodiments, the at least one secondary absorbent layer comprises a paper-based material. In embodiments, the at least one secondary absorbent layer is interposed between a first primary absorbent layer and a second primary absorbent layer. In embodiments, the disposable absorbent area further comprises at least one of a specialized inner layer and a specialized outer layer. The specialized outer layer can comprise a biopolymer having a barrier property. The specialized inner layer can comprise a functional additive. In embodiments, the article of manufacture further comprises a reusable outer shell that positions the disposable absorbent area in proximity to an anatomically advantageous area within the article of manufacture. In embodiments, the article of manufacture is selected from the group consisting of diapers, incontinence pads, feminine hygiene products, pet litter, and pet training pads. The article can be a personal care product, which can be selected from the group consisting of diapers, adult incontinence products, fluid absorption pads, and feminine hygiene products. The article can be intended for a medical use, which can be selected from the group consisting of wound treatment, blood coagulation, treatment of a skin condition, surface application of a medical or wellness treatment, and transdermal dissemination of a pharmaceutical treatment.

### BRIEF DESCRIPTION OF FIGURES

FIG. 1 depicts schematically a cross-section of a layered absorbent area.

FIG. 2A depicts schematically a cross-section of a multilayered absorbent area.

FIGS. 2B and 2C each depict schematically top views of a multilayered absorbent area such as has been shown in FIG. 2A.

### DETAILED DESCRIPTION

### 1. Absorbent Materials Formed from Renewable Resources

Disclosed herein, in embodiments, are absorbent materials comprising bio-based hydrogels formed from renewable resources. Such materials are understood to be naturally derived, biodegradable, and environmentally sustainable. While the term “sustainable” has many meanings in current usage, it generally refers to being able to maintain a condition or a set of behaviors at a certain rate or level over a period of time; as used in the environmental context, the term generally refers to avoiding the depletion of natural resources and maintaining an ecological balance. Advantageously, the materials described herein are intended to contribute to this desirable objective of sustainability, defined by the EPA as “to create and maintain conditions, under which humans and nature can exist in productive harmony, that permit fulfilling the social, economic, and other requirements of present and future generations.” (Executive Order 13514 (2009) Federal Leadership in Environmental, Energy, and Economic Performance).

The absorbent materials disclosed herein contribute to sustainability because they are naturally derived and biodegradable, in contrast to those absorbent materials derived from non-renewable sources such as petroleum (i.e., conventional superabsorbent polymers or SAPs). As used herein, a “conventional superabsorbent polymer” is a polyacrylate superabsorbent polymer, for example, a crosslinked polyacrylate superabsorbent polymer. In embodiments, the absorbent materials disclosed herein can be used in personal and pet care articles, such as baby diapers, adult incontinence pads, feminine hygiene products, and animal litter. In embodiments, the articles comprising the absorbent materials disclosed herein demonstrate advantageous performance characteristics such as fluid absorption capacity, and fluid absorption rate (wicking speed), and rewetting, where one or more than one of these performance characteristics are within a commercially acceptable range. For example, one or more than one of these performance characteristics can be within about 80% of the same or a similar characteristic exhibited by articles comprising conventional superabsorbent polymers, or can be within about 85% of the same or a similar characteristic, or within about 90% of the same a similar characteristic. In another example, one or more than one of the advantageous performance characteristics exhibited in the articles comprising the absorbent materials disclosed herein is within about 80% of the same characteristic, or is within about 85% of the same characteristic, or within about 90% of the same characteristic as such characteristic is exhibited by articles comprising conventional superabsorbent polymers, e.g., the fluid absorption capacity, and fluid absorption rate (wicking speed), and/or rewetting of the adsorbent material of the present invention can be within about 80%, 85%, or 90% of the fluid absorption capacity, and fluid absorption rate (wicking speed), and/or rewetting, respectively, of an adsorbent material comprising conventional superabsorbent polymers. In embodiments, the cumulative performance of all the performance characteristics is within a commercially acceptable range, even though none of the performance characteristics in isolation is within at least about 80% of similar characteristics exhibited by articles comprising conventional superabsorbent polymer, or within at least about 85% of similar characteristics, or within at least about 90% of similar characteristics. In embodiments, a commercially acceptable range of performance characteristics is understood by those having ordinary skill in the art. In embodiments, the cumulative performance of the advantageous performance characteristics for articles comprising the absorbent materials disclosed herein is comparable to or superior to those of those performance characteristics exhibited by articles comprising conventional superabsorbent polymers.

In more detail, the hydrogels disclosed herein are formed from polymers demonstrating superabsorbent properties, where the term “superabsorbent” refers to the ability of the material to absorb at least ten times its dry weight of an aqueous liquid. The term “polymer” refers to a macromolecule having a degree of polymerization of at least 1000. In embodiments, a polymer can have a molecular weight of at least 1000 Daltons. A polymer may be a homopolymer, copolymer, terpolymer, or other macromolecular grouping that would be recognized as such by artisans of ordinary skill; a polymer may be linear, branched, and/or crosslinked. Superabsorbent polymers, such as are disclosed herein, are understood when crosslinked to absorb fluid via osmosis to form a relatively durable gel, which may be termed a “hydrogel.”

Advantageously, the superabsorbent polymers (or SAPs) disclosed herein are produced in whole or in part by living organisms, or are derived from living organisms or other renewable resources. For the purposes of this disclosure, renewable resources are those products of nature that are replenished, restored, or regrown within a fairly short time frame, for example, within less than 100 years. By contrast, natural resources such as petroleum, coal, minerals from the earth, and peat take longer than 100 years to replenish themselves, so are not included as renewable resources. Renewable resources may be replenished naturally or via agricultural techniques or other engineering techniques. Agricultural techniques include the cultivation of the land and the husbandry of animals, fish, or other living organisms (bacteria, algae, fungi, and the like). As examples and without limitation, renewable resources include plants, animals, fish, bacteria, fungi, and forestry products or byproducts, any of which may be naturally occurring, hybridized, or genetically engineered, and any of which can be sourced from their primary natural environment, or from an engineered environment such as a culture or hydroponic horticulture. Materials derived from such renewable resources can also be termed “bio-based” materials.

Advantageously, the bio-based SAP materials disclosed herein are biodegradable and compostable. Biodegradation is the mineralization (i.e., the degradation of a material to its mineral components, which may be simply carbon dioxide and water, or which may include other minerals such as nitrates, sulfates, halogens, etc., depending on the makeup of the material itself) resulting from the action on the material of microorganisms such as bacteria and fungi. A material that can be decomposed by biodegradation can be termed “biodegradable,” as that term is used herein. By contrast, many synthetic materials such as petroleum-derived plastics are understood to be resistant to biodegradation, limited in part by their molecular weight, chemical structure, water solubility. Importantly, these synthetic materials are xenobiotic, i.e., not present in the environment until recently, so that they were not included in the evolutionary processes that formed the metabolic pathways among microorganisms that could result in their degradation. By contrast, naturally occurring bio-based polymers, as are found in or derived from living organisms, evolved in symbiosis with microorganisms that can degrade them. Biodegradation of bio-based polymers therefore takes place relatively rapidly, especially for those bio-based polymers that have hydrolysable linkages in their backbones, including cellulose, hemicellulose, and various polysaccharides.

Biodegradation begins with the encounter of the microorganisms with the bio-based material, following which the microorganisms secrete various extracellular enzymes that depolymerize the bio-based material. Once the polymer is reduced to a size that renders its fragments water-soluble, these fragments can be taken up by the microorganisms and subjected to the microbial metabolic pathways that mineralize the fragments. Other non-biotic chemical processes such as chemical degradation and photo-oxidation can take place before or during these microbial-driven processes as part of the biodegradation process. The term “compostable” is often used interchangeably with biodegradation, although a number of more specific legal definitions exist to differentiate this term from biodegradation. For example, the European standard EN13432 defines minimum standards for packaging materials to be deemed compostable: 1) disintegration (i.e., fragmentation and loss of visibility in the final compost, with at least 90% of the composted materials able to pass through a 2×2 mm sieve), whereby the residue from the original material <10% of the original mass after 3 months; 2) biodegradability, with the 90% of the composted material converted into CO₂ through the action of microorganisms within 6 months; 3) absence of negative effects of the composting process; 4) heavy metals and fluorine below certain specified amounts.

### 2. Absorbent Materials Comprising Bio-Based Hydrogels

In embodiments, the absorbent materials disclosed herein comprise bio-based hydrophilic and water-swellable polymers that form hydrogels. As used herein, the term “bio-based” refers to a material that can be obtained from renewable resources derived from living organisms, such as those materials formed by plants, microorganisms or animals. As used herein, the term “natural” also refers to such materials. A hydrogel is understood to be a three-dimensional network of hydrophilic polymers that can swell in water and hold a relatively large amount of water while maintaining the three-dimensional structure, due to the chemical or physical interaction of the component polymer chains. As used herein, the term “hydrogel” refers to a relatively water-insoluble gel that is formed from inclusion of water within a matrix of water-swellable materials. Hydrocolloids are colloidal systems with hydrophilic polymers dispersed in water; hydrocolloids can assume different states, whether sol or gel, depending on the component materials and the amount of available water. Hydrocolloids can also alternate between sol and gel states, for example if exposed to head or other physical or chemical agents.

Certain hydrogels can be formed from cross-linked or entangled networks of linear homopolymers, linear copolymers, or block or graft copolymers. Other hydrogels can be formed as interpenetrating networks, physical blends, or hydrophilic networks stabilized by hydrophobic domains. In other instances, hydrogels can be formed as polyion-multivalent ion complexes, or polyion-polyion complexes, or hydrogen-bonded complexes. Hydrogels can be reversible (physical) hydrogels or permanent (chemical) hydrogels. Physical hydrogels include: simple entanglement systems, in which the water-containing polymeric network is held together by molecular entanglements or crystallites; ion-mediated networks, in which the network is stabilized by interaction between oppositely charged polyelectrolyte and multivalent ions; and thermally induced networks that form three-dimensional structures in response to heating or cooling. Chemical hydrogels are mainly supported by covalent bonds, including bonded structures like cross-linked polymers or copolymers, or polymerized interpenetrating networks. Hydrogels can also be formed by crosslinking or entanglements that take place in response to external stimuli, including application of light and changes in temperature. Light stimulus is especially advantageous for crosslinking applications because its delivery is easy to regulate and quantify. Lights can be switched on and off, allowing the dose to be controlled precisely to achieve the desired functional effects. Moreover, light wavelength can be selected specifically to produce desired properties in the resultant hydrogel. Ultraviolet light exposure is advantageous, while other wavelengths can be selected as appropriate.

Materials used to form bio-based hydrogels include natural hydrophilic polymers that absorb significant amounts of water or aqueous fluids (including but not limited to body fluids) in a relatively short period of time. In other words, such polymers are water-swellable. A water-swellable polymer can be dispersed in a liquid phase such as water, wherein it can imbibe that water; if crosslinked, the water-swellable polymer in the aqueous milieu can form a hydrophilic polymeric network that ensnares the liquid within the network through surface-tension effects and hydrogen bonding, thus forming a gel. A crosslinked water-swellable polymer, such as are disclosed herein, soak up water, complexes with it, or otherwise binds to it to form a three-dimensional network, known as a hydrogel. Hydrophilic groups on the polymers can account for the avid hydrophilicity of a water-swellable polymeric network. Water-swellable polymeric networks may range from being mildly absorbing, typically retaining 10-30 wt.% of water within their structure, to super absorbing, where they retain many times their weight of aqueous fluids. When they form into a stable three-dimensional gel structure, this structure is termed a hydrogel, as described above. In embodiments, a hydrogel comprises at least 10% water.

Water-swellable polymers and the hydrogels that they form are themselves or can be derived from natural materials. For example, natural water-swellable polymers include anionic polymers like alginate and carrageenan, and cationic polymers like chitosan, as well as neutral polymers. In embodiments, the water-swellable polymeric materials can comprise naturally-derived hydrocolloids that comprise high molecular weight hydrophilic polymers whose polar or charged functional groups render them soluble in water and further impart water-swellable properties. Naturally-derived water-swellable polymers include polysaccharide polymers such as dextran, dextrin, agarose, cellulose, starches, and derivatives thereof. Polysaccharides have additional advantages of biodegradability and well-recognized, regulatory-friendly acceptance for personal care and other health and wellness applications. In more detail, suitable hydrogels and hydrocolloids for forming bio-based absorbent materials can include, without limitation, xanthan gum, pectin, amylopectin, carrageenan (or including without limitation kappa, iota or lambda carrageenans), alginate and alginates (including without limitation derivatives such as propylene glycol alginate), agar-agar, cellulose gum, celluloses (such as carboxyalkyl celluloses, including but not limited to carboxymethylcellulose, hydroxyethyl cellulose, carboxymethyl hydroxyethyl cellulose and the like), pectin ester, gums such as gellan gum, guar gum and guar derivatives, gum Arabic, locust bean gum, diutan, welan, tarn, olibanum, karaya, ghatti, dammar, tragacanth gum, or modifications or mixtures of any of the foregoing. In embodiments, polysaccharides such as are described herein are especially advantageous for their swellable properties when in high molecular weight or high viscosity formulations. For example, polysaccharides having molecular weights of up to about 90,000 Daltons, up to about 100,000 Daltons, up to about 150,000 Daltons or higher molecular weights up to about 1,000,000 Daltons, or molecular weights between about 50,000 and about 2,000,000 Daltons are useful for forming hydrogels. In certain embodiments, lower molecular weight polymers are useful, such as cellulose ether polymers with molecular weights in a range from about 10,000 to about 100,000 Daltons, such as about 10,000 Daltons.

Mixtures of naturally derived hydrogels can also be formed. As examples, polysaccharides such as starch, modified starches, amylose, modified amylose, celluloses such as cellulose ethers and cellulose esters, chitosan, modified chitosan, chitin, modified chitin, gelatin, konjac, modified konjac, fenugreek gum, modified fenugreek gum, mesquite gum, modified mesquite gum, aloe mannans, modified aloe mannans, oxidized polysaccharides, sulfated polysaccharides, cationic polysaccharides, and the like, can be used alone or in combination with other such materials, in any ratio. In embodiments, a mixture of cellulose ether polymers can be prepared for use as an absorbent material. For example, a formulation comprising hydroxyethyl cellulose and hydroxypropyl methylcellulose can be prepared, optionally in combination with a plasticizer such as glycerol and a surfactant (e.g., capryl glucoside, hexyl glucoside, and the like), to form an absorbent polymer. High molecular weight or low molecular weight ratios of the ingredients of the formulation can be adjusted to provide advantageous properties. In an embodiment, a formulation comprising about 0.5% to about 5%, or about 0.5% to about 4%, or about 1% to about 2% of the naturally derived hydrogels can be prepared to include surfactants and plasticizers, with ratios of the hydrogel portion to the surfactant ranging from about a 1:1.5 ratio of hydrogel: surfactant to about a 2:1 ratio of hydrogel: surfactant, for example, a 1:1 ratio of hydrogel: surfactant, and with ratios of the hydrogel portion to the plasticizer ranging from about a 95:5 ratio of hydrogel: surfactants to about a 99:1 ratio of hydrogel: surfactant. In a preferred embodiment, the hydrogels comprise about 1.2% of the formulation, with a hydrogel: surfactant ratio of about 1.5:1 and a hydrogel:glycerol ratio of about 95:5.

Polysaccharide polymers are advantageous for forming absorbent materials. As an example, xanthan gum (XG) can be used. XG is an anionic polysaccharide resistant to a broad range of changes in temperature, pH, and salinity. XG can form rigid helical structures due to the availability of hydrogen bonds between its trisaccharide sidechains and its polymer backbone. Consequently, the randomized spatial orientation of these rigid helices renders them capable of high swelling performance when crosslinked. As another example, alginic acid polymers or sodium alginate can be used. Alginic acid is a linear copolymer with homopolymeric blocks of (1→4)-linked β-D-mannuronate (M) and α-L-guluronate (G) residues, respectively, covalently linked together in different sequences or blocks. The monomers may appear in homopolymeric blocks of consecutive G-residues (G-blocks), consecutive M-residues (M-blocks) or alternating M and G-residues (MG-blocks). Note that α-L-guluronate is the C-5 epimer of β-D-mannuronate. In embodiments, biodegradable synthetic polymers can also be used, such as polyvinyl alcohol, polyvinyl acetate, poly lactic acid, polyglycolic acid, polylactic-co-glycolic acid, etc., or mixtures thereof. Polysaccharide polymers that are capable of taking up at least 50 times, at least 100 times, at least 300 times, at least 500 times, at least 800 times, at least 900 times, or at least 1000 times their weight in water are particularly useful.

High molecular weight swellable polymers may be especially advantageous for swelling due to their ability to create a highly entangled, porous network, and may be preferable, in embodiments, to smaller molecular weight molecules. In other embodiments, creating a highly entangled network from smaller molecular weight polymers (or creating an “ultra” high molecular weight polymer network from already high molecular weight polymers) can greatly increase swelling capacity. Polymeric mixtures can be used to create this ultra-high molecular weight network through charge-charge complexation. In embodiments, highly branched, charged, swellable polymers, can be used and mixed with an oppositely charged polymer. The branched components can then be linked up, forming a stable network. In embodiments, a plasticizer (as described in more detail below) such as glycerin, can be used to promote absorption and keep the apertures, pores, or other channels between the two oppositely charged polymers propped open. As used herein, the term glycerol refers to a pure form of the glycerol molecule (1,2,3-propanetriol)s, while glycerin refers to a formulation containing about 95% glycerol. A plasticizer such as glycerin exerts its advantageous effects due to the presence of glycerol in the glycerin; it is understood that using unadulterated glycerol as a plasticizer would be similarly advantageous.

A small amount of any neutrally charged plasticizer, oligomer, or polymer may also be used in between the polymeric complexes to prevent too much clumping or precipitation. It is envisioned that the network thus formed can be sufficiently stable that crosslinking is not necessary, or that crosslinking can be minimized.

In embodiments, a positively charged polymer can be used as the majority component, and a negatively charged polymer can be used more scarcely and provide linkages. These can be switched, with the negatively charged polymer doing the swelling and the positively charged polymer creating the linkages. Varied amounts can be used, such as ratios of 90: 10 to 95:5 swellable polymer to linking polymer. In embodiments, the major polymeric component is highly swellable, and in other embodiments both component polymers are highly swellable. In an embodiment, cationic starch can be used in combination with an oppositely charged polymer such as CMC, alginate, pectin, and the like.

In embodiments, mixtures of more linear polymers can be prepared to create a single high molecular weight entangled polymer that has a different polymeric arrangement than polymers comprising linked branched components. In embodiments, a small amount of positively charged polymer, such as chitosan, can be used, with a larger amount of a cost-effective negatively charged polymer, such as CMC, alginate, pectin, and the like. A plasticizer, such as glycerin, can be used the lubricate and prop open the chains and to allow for more water uptake and to prevent clumping or precipitation. The polymer charges can be switched, but varied amounts can be used such as ratios of 90:10 to 95:5. In embodiments, the major polymeric component is highly swellable, and in other embodiments both component polymers are highly swellable.

In embodiments, other additives can be included in the water-swellable polymer formulation (whether such a formulation includes only a single hydrogel-forming polymer or whether it includes mixtures of such polymers) to improve performance attributes such as fluid absorption (capacity), fluid absorption rate (wicking speed), and rewetting. As an example, the addition of glycerin or similar compounds to the formulation can improve certain aspects of performance, such as wicking speed. In embodiments, glycerin can be added in small amounts, for example, in an amount between about 0% to about 30%, or between about 0% to about 20%, or between about 5% and about 15%, or between about 5 to about 10% of the polymer add-on. In embodiments, glycerin can be added in small amounts, for example in an amount between about 0% to about 5%, or between about 10% to about 15%, or between about 15% and about 20%, or between about 20% or about 25%, or between about 25% and about 30%. As an example, glycerin can be added to a XG formulation in an amount of about 10%: a coating formulation comprising 10% XG polymer and 1% glycerin can improve swelling performance and improve coating consistency with reduced flakiness and enhanced ductility.

Water uptake in the bio-based hydrogel can be enhanced by adding a plasticizer material. As used herein, the term “plasticizer” refers to a low volatility, low molecular weight organic substance that is added to a polymer to improve its physical/handling properties such as its flexibility, flow and/or thermoplasticity without changing its chemical properties, for example by decreasing its viscosity, altering its glass transition temperature (T_{g}), or modifying the elastic modulus of the polymer composition. Without being bound by theory, it is understood that a plasticizer can improve the mechanical properties of a polymer system by interacting with the polymeric chains to facilitate their physical interaction with each other or by occupying the space between the polymeric chains to increase the free volume between them, allowing them to slide and rotate more freely, thereby decreasing the T_{g}, and melt viscosity.

Plasticizers useful for improving the water uptake in a bio-based hydrogel can include one or more types of low molecular weight, hygroscopic materials, for example small molecules or oligomers. Small molecule examples include polyols such as glycerol or glycerin, ethylene glycol, diethylene glycol, triethylene glycol, tetraethylene glycol, hexylene glycol, butylene glycol, polyethylene glycol, propylene glycol, tripropylene glycol, sorbitol, mannitol, maltitol, xylitol, erythritol, isomalt, and the like. Polymeric polyols such as polydextrose can be used as well. As other examples, suitable plasticizers can include other hygroscopic materials such as acetin, glyceryl triacetate, urea, collagen, etc. Oligomers can also be used as plasticizers, such as dimers, trimers, tetramers, etc. Examples of oligomers include glucose oligomers, such as dextrose, maltose, maltotriose, maltotetraose, and the like. Cellulose oligomers can also be used, such as cellotriose, cellotetraose, and the like. Any of these plasticizer materials can be used alone or in combination with one another in any ratio, such as in an amount between about 0% to about 30%, or between about 0% to about 20%, or between about 5% and about 15%, or between about 5 to about 10% of the polymer add-on. In embodiments, plasticizers can be added in small amounts, for example in an amount between about 0% to about 5%, or between about 10% to about 15%, or between about 15% and about 20%, or between about 20% or about 25%, or between about 25% and about 30%. Plasticizer additives can increase the rate, amount and retention of the bio-based hydrogel matrix, allowing, for example, more rapid uptake of a body fluid such as urine, a larger capacity for holding a volume of body fluid, and/or a longer period of retention of the body fluid within the hydrogel matrix. In embodiments, a plasticized bio-based hydrogel can be formed from a highly viscous solution of the selected polymer or polymers, comprising about 1% to about 50% polymer(s), or about 5% to about 25% polymer(s), with one or more plasticizers added to it in amounts such as between about 0% to about 30%, or between about 0% to about 20%, or between about 5% and about 15%, or between about 5 to about 10% of the polymer add-on, or with plasticizers added to it in other small amounts, for example in an amount between about 0% to about 5%, or between about 10% to about 15%, or between about 15% and about 20%, or between about 20% or about 25%, or between about 25% and about 30%.

After the plasticized hydrogel is formed, it can, optionally, be crosslinked. Crosslinkers can be added during the manufacturing process, or crosslinking precursors can be added that are intended to form after the hydrogel is exposed to the body fluid, for example urine. In embodiments, the hydrogel can be crosslinked on the surface, either during the manufacturing process or secondarily (for example, upon exposure to a body fluid). In other embodiments, the hydrogel can be crosslinked internally, partially or throughout its substance. In yet other embodiments, a combination of surface and internal crosslinking can be employed.

In embodiments, surface crosslinking can be advantageous for preventing gel blocking. Gel blocking occurs when hydrogel particles, as can be used in an absorbent pad of a diaper or personal care article, become engorged and enlarged after absorbing a certain amount of liquid, following which the hydrogel particles deform, shift, and clump onto each other, blocking voids in the supportive matrix of the absorbent pad and inhibiting further transmission of liquid to other parts of the absorbent pad.

However, surface crosslinking is designed to be relatively weak so that it does not constrain bead swelling, but as a consequence the surface crosslinks might not have sufficient strength to withstand the stresses of swelling or the stresses associated with load bearing when the article is being worn, so rewetting may occur easily. In embodiments, internal crosslinking can be advantageous in addition to or instead of surface crosslinking. Increasing the internal crosslinking can increase the gel strength of the liquid-saturated absorbent particles, thereby improving the structural integrity of the absorbent layer.

In embodiments, crosslinking can be engineered to achieve particular results, balancing a number of factors, including preventing gel blocking, maintaining adequate structural stability, and providing sufficient absorptive capacity and rate of absorbency. In embodiments, absorbent particles can be formed into shapes that facilitate the optimal balance of these factors. For example, in embodiments, particles can be formed that are elongate, or that have a high aspect ratio, in order to optimize absorbency while still maintaining structural stability and interfering with gel blocking.

Crosslinkers that can be added to the hydrogel include covalent crosslinkers and ionic crosslinkers. Covalent crosslinkers include bi and multifunctional epoxies, citric acid, butanetetracarboxylic acid, poly(methyl vinyl ether-alt-maleic anhydride), polymeric methylene diphenyl isocyanate, poly(ethylene glycol) and diglycidyl ether. Ionic crosslinkers include salts with divalent ions, such as calcium chloride, magnesium chloride, calcium citrate, magnesium citrate, calcium acetate, magnesium acetate, etc. Monovalent or other multivalent salts may be used as well. Catalysts can be added to accompany specific crosslinkers, as would be understood by artisans of ordinary skill. Crosslinking can also be performed secondarily, due to exposure to a body fluid such as urine. It is understood that urine contains divalent cations that act as secondary crosslinking agents capable of crosslinking certain anionic hydrogel polymers, for example, alginate, carboxymethyl cellulose, and the like.

Additional examples of crosslinking agents include polyglycidyl ether compounds, haloepoxy compounds, polyaldehyde compounds, polyhydric alcohol compounds, polyamine compounds and polyisocyanate compounds. Multifunctional epoxides are particularly advantageous, for example, polyglycidyl ether compounds such as ethylene glycol diglycidyl ether, propylene glycol diglycidyl ether, glycerol-1,3-diglycidyl ether, glycerol triglycidyl ether, triglycidyl ethers of propxylated glycerin, polyethylene glycol diglycidyl ether and 1,6-hexanediol diglycidyl ether, and the like. Examples of haloepoxy compounds include epichlorohydrin and α-methyl epichlorohydrin. Examples of polyaldehyde compounds include glutaral-dehyde and glyoxal. Examples of polyhydric alcohol compounds include glycerol, ethylene glycol, diethylene glycol, propylene glycol, polyethylene glycol, polypropylene glycol, diethanol amine and triethanol amine. Examples of polyamine compounds include ethylene diamine, diethylene triamine, triethylene tetramine, tetraethylene pentamine, polyamide resin as a reactant of polyamine and aliphatic polybasic acid and polyamide polyamine epichlorohydrin resin. Examples of polyisocyanate compounds include toluene diisocyanate, and hexamethylene diisocyanate.

If epoxy crosslinkers are used, bases, tertiary ammonia, and quaternary ammonium catalysts can be added to reach appropriate crosslinking conversions. Depending on the source and grade of the polysaccharide used, varying amounts of crosslinker may be used. To accomplish this, a crosslinker can be selected that has flexible and extendible arms between crosslinking sites, so that the interior polymer chains within the polysaccharide coating can expand upon imbibition of liquid, allowing the matrix to retain liquid and swell.

In embodiments, a crosslinker formulation can comprise multifunctional epoxies with oligomeric arms. Such bulky crosslinkers are slow to diffuse, especially in a viscous solution used to produce an absorbent particle. This high viscosity layer results in reduced crosslinker diffusivity, which directs the reaction predominantly to the surface of the layer, while its interior remains unconstrained and uncrosslinked. An unconfined interior within the coating layer permits its facile expansion/swelling. In yet other embodiments, crosslinking is not required or advantageous, and thus can be omitted.

### 3. Shaping Absorbent Materials for Use in Articles of Manufacture

Absorbent materials formed from the bio-based hydrogel formulations disclosed herein can be formed into any useful shape, including, but not limited to, beads, pellets, strands, fibers, chips, sheets, plates, and the like. The shape of the absorbent material can be engineered for a specific application by directing the absorbent material composition as described above through an appropriate shape-forming apparatus, which can form the composition into a three-dimensional configuration that is consistent with ultimate formation into the desired pre-designated useful shape. A shape-forming apparatus can be an extruder, an electrospinner, a slot-die, a mold, or any other apparatus available in the art for fashioning a liquid formulation into a solid or semi-solid structure having a defined three-dimensional configuration consistent with the predesignated shape. As used herein, the term “liquid” refers to a material that assumes the shape of the container in which it is held and is not solid; liquids include semi-fluid materials such as gels that are flowable but are highly viscous. By contrast, a solid is a state of matter that retains its shape and density when not confined. A semi-solid material is a non-flowable material that does not pour easily but is less dense and rigid than a solid. A non-flowable gel that remains in its formed shape can be considered a semi-solid. As described in more detail below, a shape-forming apparatus can transform a liquid or semi-fluid material into a solid or semi-solid material having a preselected shape. In embodiments, the shape-forming apparatus itself suffices to form the liquid or semi-fluid material into the predesignated shape for the solid or semi-solid material. In other embodiments, the shape-forming apparatus forms the liquid or semi-fluid material into a three-dimensional configuration that can then be dried to produce the predesignated shape for the solid biodegradable absorbent material.

Shapes for the absorbent materials are selected based on the commercial need for absorbent articles: for example, flat strands or randomly shaped particles may be more useful for diaper manufacturing as opposed to rounded beads or strands that can accumulate in the dependent regions of the diaper. As another example, strands or fibers may align themselves better with other filler material such as wood pulp or fluff pulp for applications like animal litter in which softness and “paw feel” are important. In embodiments, elongated form factors, especially those having a high aspect ratio, are less likely to contribute to gel block. Thus, an elongated shape can be advantageous for preventing gel block in absorbent articles, in addition to the crosslinking strategies mentioned previously. A multitude of shapes can be engineered for absorbent materials, depending on the needs in a particular absorbent article. For example, in embodiments, the absorbent material can be formed as long, flattened strands with a lower aspect ratio or can be formed as flattened sheets, instead of as particles, cylinders, or beads. Molds, for example, can form a liquid absorbent material substrate into a predesignated three-dimensional structure.

In one version of the manufacturing process, the high viscosity hydrogel-forming polymer and plasticizer mixture can be metered into and pumped out of a drop tower, which acts as a shape-forming apparatus by allowing the formation of droplets from the fluid pushed through the orifices in the drop tower head, analogous to the formation of water droplets from shower head. As the droplets descend from the drop tower head, they solidify and assume a symmetrical shape. The shape and hardness of the droplets can be adjusted as they solidify by exposure to heat and to countervailing air currents. For example, an air blower at the bottom or on the sides of the droplet stream can provide air currents and/or heating to slow down droplet descent, adjust droplet size, accelerate gel hardening, or facilitate crosslinking. In processing methods that intend to augment crosslinking, droplets can be expelled from a drop tower head as described above and directed into a bath containing crosslinking solution. The resulting crosslinked droplets can be removed from the bath by filtration, centrifugation, or other methods familiar in the art. Hydrogel particles produced by the techniques described above can be further cut or processed to obtain desired sizes or shapes.

In another version of the manufacturing process, extruders can be used as shape-forming apparatuses. In an exemplary embodiment, a homogenous high viscosity hydrogel mixture comprising the selected natural polymer(s) and plasticizer(s) is produced in a mixing tank, with crosslinkers added as needed and with other additives optionally included. An advantageous formulation comprises a mixture of hydroxyethyl cellulose (HEC) and hydroxypropylmethyl cellulose (HPMC), in combination with a plasticizer such as glycerol or glycerin and a surfactant such as capryl glucoside or hexyl glucoside; such a formulation can have a HEC:HPMC ratio ranging from about 95:5 to about 70:30, for example 80:20, or a range from about 95:5 to about 60:40. The absorbent polymers in the formulation are added in an amount between about 1% and 2% by weight, for example about 1.2%. The ratio of absorbent polymers to surfactant is about 1.5:1, and the ratio of the absorbent polymers to glycerol is about 95:5.

To prepare an exemplary formulation, glycerol can be added to water and stirred magnetically for about 5 minutes, following which a surfactant such as capryl glucoside or hexyl glucoside can be added, with further mixing as can be performed by an overhead mixer. The selected polymer (e.g., CMC or HPMC), or mixture of polymers as described above, can be added to the solution and mixed for a specified period of time (for example, at 300 rpm for 5-20 min, with the speed then decreased to 150 rpm for another 6-10 hours of mixing). Crosslinkers can be added at any stage in the mixing process, for example by adding non-immediate crosslinkers; in embodiments, non-immediate crosslinkers can replace ionic ones (which take effect immediately), so that crosslinking can be performed at any stage of the reaction, while in other embodiments ionic crosslinkers can be added at the end of the reaction and allowed to take effect immediately. If a catalyst is employed to facilitate crosslinking, the crosslinker can be added at any appropriate point, and the catalyst would be added at the end of the reaction period.

After the ingredients are mixed, the resultant mixture is pumped through an extruder with devolatilization capacity, allowing extra water to be evaporated and producing an extruded strand of the material. Crosslinkers and/or catalysts that are activated upon heating can be included in the mixture or added during the extrusion process, so that the formed, extruded material will begin to crosslink upon passage through the extruder. Heating elements/equipment can be added after extrusion to heat the extruded material in order for full crosslinking to occur. The crosslinking and/or the heating of the extruded material can solidify it so that it assumes and retains the desired predesignated shape.

In another exemplary version of the manufacturing process, fibers or strands can be formed by electrospinning. In such a process, a homogeneous high viscosity mixture comprising the selected natural polymer(s) and plasticizer(s) is produced in a mixing tank, with crosslinkers added as needed and with other additives optionally included. A formulation useful for this purpose comprises a mixture of hydroxyethyl cellulose (HEC) and hydroxypropylmethyl cellulose (HPMC), in combination with a plasticizer such as glycerol or glycerin and a surfactant such as capryl glucoside or hexyl glucoside; such a formulation can have a HEC:HPMC ratio ranging from about 95:5 to about 70:30, for example 80:20, or a range from about 95:5 to about 60:40. The absorbent polymers in the formulation are added in an amount between about 1% and 2% by weight, for example about 1.2%. The ratio of absorbent polymers to surfactant is about 1.5:1, and the ratio of the absorbent polymers to glycerol is about 95:5. A preferred formulation comprises HEC and HPMC polymers at a concentration of 1.2% of the total solution, with a ratio of absorbents to glycerol of about 95:5, and with a ratio of absorbents to surfactants of about 1.5:1. After the solution has been mixed, it can be introduced into a conventional electrospinning machine, for example by flowing it through a plastic tube into a solution reservoir for the electrospinning apparatus. The solution is then fed from the solution reservoir to a needle point in the electrospinning machine. A high voltage is produced in the needle tip, and the solution is pumped into the needle and out of the tip to reach a collector plate below. The collector plate is typically a grounded aluminum plate. Due to the difference in electrical potential between the needle tip and the collector plate, the polymer solution is spun out of the needle tip to create an array of fibers on the plate, with the fiber dimensions ranging from nanometers to micrometers in diameter. These fibers can be arranged as a randomized polymer network that can be formed into other shapes, for example as an absorbent sheet. To prepare the final shaped product from the extruded or electrospun material for use in articles of manufacture, the hydrogel strands resulting from this process can be solidified, forming dry, hardened, elongate hydrogel strands that can be cut into elongate shapes, ovoid shapes, flat shape, or any regular or irregular bead, fiber, strand, sheet, or chip shape desired. Desired bead or strand diameter would likely be 1 to 3 mm for use in absorbent personal care items, or larger (e.g., 5-10 mm) for use in pet absorbent products like animal litter. While the process above has been described with reference to forming beads or elongate strands, it is understood that final shapes for the absorbent particles can be engineered to achieve specific purposes, for example decreasing the degree of gel block that occurs in an absorbent article like a diaper, or providing a wide, flat absorbent surface, as in a flattened absorbent sheet.

As another example, the high viscosity hydrogel-forming polymer and plasticizer mixture can be prepared as sheets. A flattened structure like a sheet can be formed in a mold or by an extrusion process, in either case allowing the liquid absorbent material structure to enter the shape-forming apparatus and to be confined to or spread out upon a surface of the apparatus to solidify, for example, to dry into a solid, flattened sheet-like structure. Using a mold permits adding other design features to the sheet, such as patterns, apertures, variations of thickness, formed features, or other shape variations.

Sheets formed of the absorbent material can be used individually in an absorbent article, or a plurality of sheets can be incorporated into the article. Sheets can be of any dimension or thickness, and can have areas of varying thickness or patterned variability of thicknesses. Sheets can be embossed with patterns advantageous for the particular absorbent article, for example to promote enhanced wicking and/or to mitigate gel blocking. Sheets can be patterned with cutouts or other void areas, for example to promote water flow, or they can be sculpted with three-dimensional channels or columns to promote wicking, for example into other absorbent layers of the same material or of other materials. In embodiments, sheets can be stacked, connected to each other, and/or wrapped in, printed onto, or otherwise integrated with layers made with other materials such as pulp or porous paper. Absorbent sheets can be used in a variety of absorbent articles, for example between layers of pulp for flattened or shaped absorbent articles such as diapers or incontinence pads, or for pet training or “potty” pads, or for animal litter such as cat litter.

In an exemplary embodiment, a sheet can be formed by preparing a formulation, such as is described above, and stirring on an overhead mixer overnight, following which it can be laid out on a silicone mat or carbon pan and placed in the oven at 45 or 70° C. for 3 – 7 hours to dry. The sheet produced thereby can be rolled with a roller to a preselected thickness before or during drying, or it can be flattened to a preselected thickness before drying, recognizing that the thickness will shrink as the sheet dries. For example, a sheet that is 2 mm thick when wet can shrink to a thickness of 0.03 mm when dry. In embodiments, wet sheets of between about 1.5 to 3.5 mm in thickness can shrink to thicknesses of about 0.2 mm to 1 mm during drying. Once the sheet on the mat or pan is dry, it can be peeled off the pan to be incorporated in an article of manufacture, as described in more detail below.

In certain embodiments, the absorbent material is not pre-shaped separately, but rather is dispensed upon a biodegradable carrier in a preselected pattern, wherein the dispenser for the fluid (e.g., liquid dispensing equipment such as a nozzle, sprayer, extruder, slot die, and the like) acts as the shape-forming apparatus. The absorbent material prepared as described above can be dispensed upon the carrier in an arrangement that is appropriate for the article of manufacture, so that the fluid being absorbed is channeled, directed, or retained advantageously. Carriers can be selected from a wide variety of biodegradable materials. In embodiments, carriers derived from wood pulp can be used, for example pulp-based products like paper-based materials or fluff pulp. Paper-based materials of different consistencies can provide different properties to an absorbent article: certain paper-based materials (e.g., such as are formed into products like paper towels) can have more absorbency, while others, such as are formed into facial or bathroom tissues offer a softer surface if the article will be contacting the skin. Paper-based materials can be produced in varying thicknesses, as is suitable for the specific article of manufacture, and can be selected so as to optimize their advantageous properties such as absorption or strength. Thinner sheets of paper-based materials can be arranged in layers, where the individual layers can be treated with the absorbent material differentially in order to absorb or wick fluid in a specific pattern. Certain carrier sheets can be prepared to be more hydrophobic, for example, and can be combined with one or more layers of other carrier sheets that are less hydrophobic. In embodiments, internal or external carrier sheets, for example sheets of paper, can be treated to have oil-, grease-, and/or water-resistant (OGWR) properties. In embodiments, such a sheet having OGWR properties or set of layered sheets having OGWR properties is positioned on or towards the externa aspect of the formed article, it can provide an external barrier that prevents the passage of fluid out of the article of manufacture into the environment.

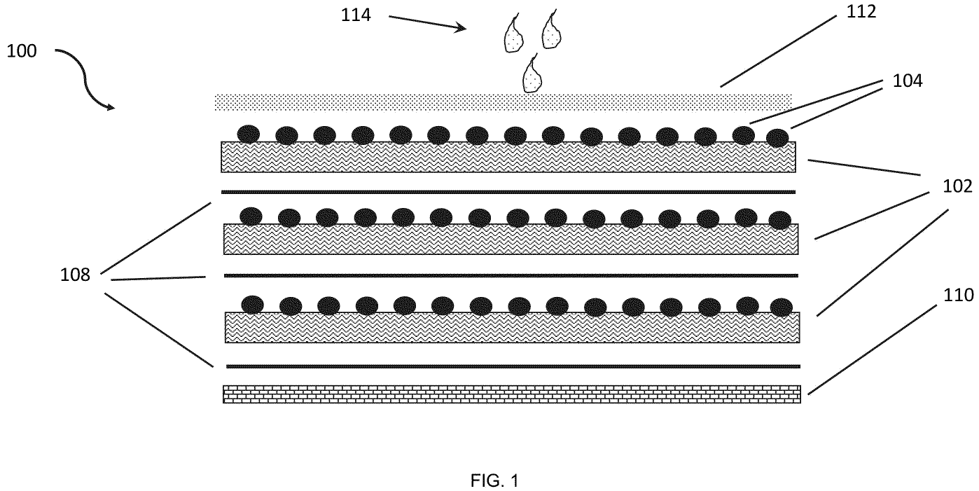

A layering arrangement of carrier sheets treated with the absorbent material as disclosed herein can be separated by sheets of the absorbent material itself to exploit the ability of the treated carrier sheets to direct fluid flow in combination with the ability of the absorbent material to absorb large amounts of fluid. An exemplary arrangement of layering to create an absorbent area in an article of manufacture is depicted in FIG. 1. FIG. 1 shows schematically a cross-section of such an absorbent area 100. As used herein, the term “absorbent area,” such as the absorbent area 100 represented by FIG. 1, refers to an absorbent structure that is suitable for use on its own as an absorbent article, or that can be integrated into more complex articles of manufacture. When incorporated into a more complex article of manufacture, the absorbent area can also be termed the “absorbent core,” depending on the relationship of the absorbent area to other features of the article of manufacture. Exemplary uses for absorbent areas such as are described with respect to this Figure are provided below.

In the embodiment of an absorbent area 100 depicted in FIG. 1, layers of biodegradable carrier material 102 are interleaved with layers of absorbent material 108. While the layering arrangement appears to include spaces between the layers, such spaces are only to allow the individual layers to be viewed more easily. While spacing between layers can be provided in the absorbent area 100, more commonly the layers are juxtaposed closely to each other. In embodiments, the layers of carrier material 102 and absorbent material 108 are compressed together to produce a thinner cross-section for the absorbent article 100. In the depicted embodiment, the layers of carrier material 102 carry on their top surface a dispersion of absorbent material 104. While the multiple areas of absorbent material 104 on the surface of the carrier material 102 appear in the Figure to be regularly spaced droplets or dots, it is understood that any patterning of dispersion for the absorbent material 104 can be arranged, whether regular or irregular, continuous or discontinuous, linear with parallel, converging, or overlapping lines, serpentine, dotted, scattered, shaped as a grid or a network, or otherwise, in order to achieve the desired objectives of the absorbent area 100. In embodiments, such absorbent material 104 dispersed on the carrier material 102 is optional. In embodiments, the layers of absorbent material 108 are optional, with one or more layers of carrier material 102 plus the optional absorbent material 104 disposed thereon providing the desired absorbency.

Methods for dispersing the absorbent material 104 on the carrier material 102 are familiar in the art. For example, a biodegradable absorbent polymer solution as disclosed herein can be dispensed from an applicator or sprayed onto the carrier material 102 when the absorbent polymer solution is still in a liquid form. In such an arrangement, optionally, the carrier material can have some hydrophobic properties, so that any fluid striking the layer is preferentially directed to the absorbent polymer arrangement instead of being non-specifically absorbed by the carrier material itself. In an embodiment, the dispersion pattern for the absorbent polymer solution is arranged in channels in order to direct a fluid in a particular direction, such as wicking preferentially through a portion of the absorbent area. The carrier material 102 bearing the liquid absorbent polymer solution on its surface can then be dried, for example in an oven at 45-70° C. for 3-7 hours, which allows the carrier material layer 102 bearing the absorbent material 104 to be juxtaposed conveniently to other components of the absorbent area, and interposed therebetween. In particular, the absorbent material 104 on the carrier material 102 can come into contact with an overlying sheet of absorbent material 108. The absorbent material 104 disposed on the carrier material 102 can be the same as or different than the absorbent material in the separate absorbent material layer 108.

While the layers of carrier material 102 (optionally provided with dispersions of absorbent material 104) and of absorbent material 108 are shown as substantially similar in the depicted embodiment of FIG. 1, it is understood that any of these layers can be different from the others in size, shape, physical properties, absorbency, and the like. Carrier materials 102 themselves can be treated, apart from being provided with dispersions of absorbent material 104. For example, the carrier material can be embossed with patterns to allow directional flow or wicking of liquid, and it can be perforated to permit liquid to flow through. In embodiments, the carrier materials 102 can contain other additives within their substance, as is described below in more detail. As well, if optional absorbent layers 108 are provided, these can be similar to each other, or can differ from each other, in keeping with the absorbency requirements of the absorbent area 100. Some layers can be thicker than others to increase absorbency. For example, including absorbent layers 108 with less absorbency towards the top of the absorbent area 100 (where it is first exposed to liquid 114) can permit the liquid to pass through the top layers of the absorbent area 100 to wick away from the skin or other sensitive surface that is in contact with the top of the absorbent area 100. To avoid trapping liquid within the absorbent area structure itself or within the carrier material 102, the layers can be otherwise engineered to permit liquid passage, for example by providing holes, slits, or other mechanical features that improve fluid flow. In embodiments, the absorbent area can be formed with a predominance of absorbent layers 108 and fewer layers of carrier material 102.

As shown in FIG. 1, optional specialized inner and outer layers can be added to the layered arrangements described above. For example, a top layer 112 of a softer material can be included as the point of contact with the skin or other sensitive surface. This top layer 112 can contain functional additives having advantageous properties, as described below in more detail: for example, additives such as plasticizers (e.g., glycerin, PEG, Pluronics, and the like) to impart product softness and superior hand-feel, or vitamins (e.g., Vitamin E) or medications to treat diaper rash or other skin conditions can be included in this inner layer. Desirably, the top layer 112 is permeable to the liquid 114 it encounters, so that the liquid 114 can be absorbed by the remainder of the absorbent area 100 without remaining in contact with the skin or other sensitive area. Whether to add a specialized top layer 112 and the nature of such a specialized top layer 112 can be determined based on the article of manufacture within which the absorbent area 100 is to be included. For example, a personal care item such as a diaper can have more use for a specialized top layer 112 close to the subject’s skin, in contrast to a pad that is designed for general use such as dog training pads or disposable medical underpads, where skin contact does not exist or is not prolonged.

A bottom layer 110 can be optionally applied to the absorbent area 100 as well. In embodiments, the bottom layer is water-resistant or waterproof, and/or it can be oil and grease resistant. Water, oil, and grease resistance can be imparted by a number of techniques familiar in the art, depending on the need in a particular article of manufacture. For example, an outer waterproof layer for a personal care item can keep any absorbed fluid from passing out of the absorbent area 100 into the environment. Such a layer can also protect the absorbent area 100 from encounters with fluids in the environment. Desirably, any special properties that are imparted to the outer layer 110 are produced by using biodegradable polymeric materials (“biopolymers”), so that the entire article of manufacture is biodegradable. As used herein, the term “biopolymer” refers to those polymers that are produced by a living organism during its lifespan. Exemplary formulations can include mixtures of biobased materials such as methyl cellulose, hydroxypropylmethyl cellulose, glycerol, and the like, with the optional inclusion of additives such as nanofibrillated cellulose and microfibrillated cellulose, as described in U.S. Pat. Application 17/834,521, filed Jun. 7, 2022, the entire contents of which are incorporated herein by reference. Such a formulation can comprise some or all of the following ingredients: methylcellulose, hydroxypropyl methyl cellulose, glycerol, and microfibrillated cellulose, for example, made available for processing into a water-resistant outer layer as a 5% solution in water. Such a solution can be sprayed on a layer of carrier material 102 or can be incorporated in a layer of carrier material to impart water resistant properties to that layer, which can then form the outer layer 110 of the absorbent area 100. A formulation comprising these ingredients can alternatively be formed as a separate sheet that can then function as a separate outer layer 110 of the absorbent area.

Examples of biopolymers having barrier properties for water resistance and/or oil-grease resistance include, without limitation, exopolysaccharides such as bacterial cellulose, kefiran, pullulan, levan, gellan, and other polysaccharides such as alginate, celluloses, carrageenan, gum Arabic, starch and plant glucomannans, such as locust bean gum, mannan, guar gum, and the like. Useful biopolymers can also include biopolyesters such as polyhydroxy-alkanoates and polylactic acid derivatives. In preferred embodiments, cellulose biopolymers can be used in formulations to provide a desired degree oil/grease or water resistance for an outer layer 110. A range of cellulose polymers exists, with the various polymers having different degrees of hydrophobicity or oleophobicity, so that a cellulose polymer can be selected to produce the desired degree of oil, grease, and/or water resistance for an outer layer 110. In embodiments, it can be beneficial to mix one or more cellulose polymers with other materials that are more hydrophobic to provide more water resistance. For example, methyl cellulose provides good oil/grease resistance, but not as much water resistance. Methyl cellulose can be mixed with another cellulose biopolymer such as hydroxypropyl methyl cellulose to provide more water resistance. In embodiments, a mixture of methyl cellulose and cellulose acetate can be provided to tune for both oil/grease resistance and water resistance properties. Cellulose acetate and lipids are some examples of additives that can be used to tune oil/grease resistant coatings to be more hydrophobic, and the combination of this with a more oleophobic material can provide both oil and water resistance.

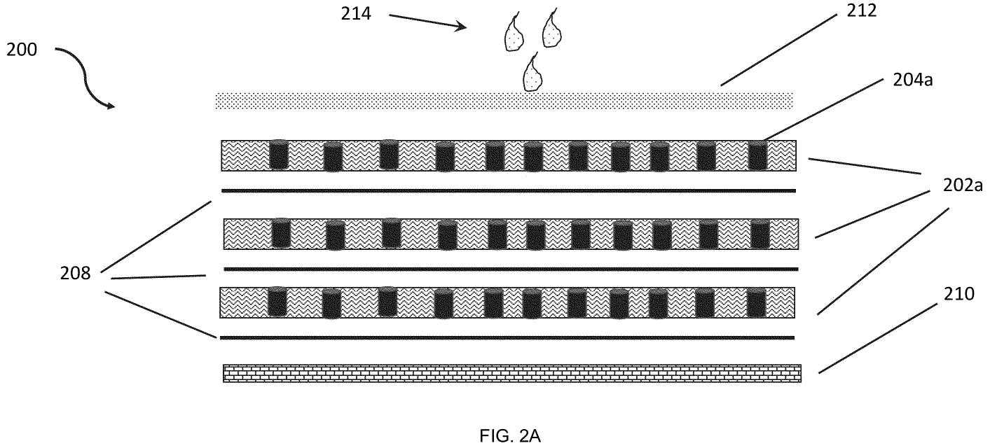

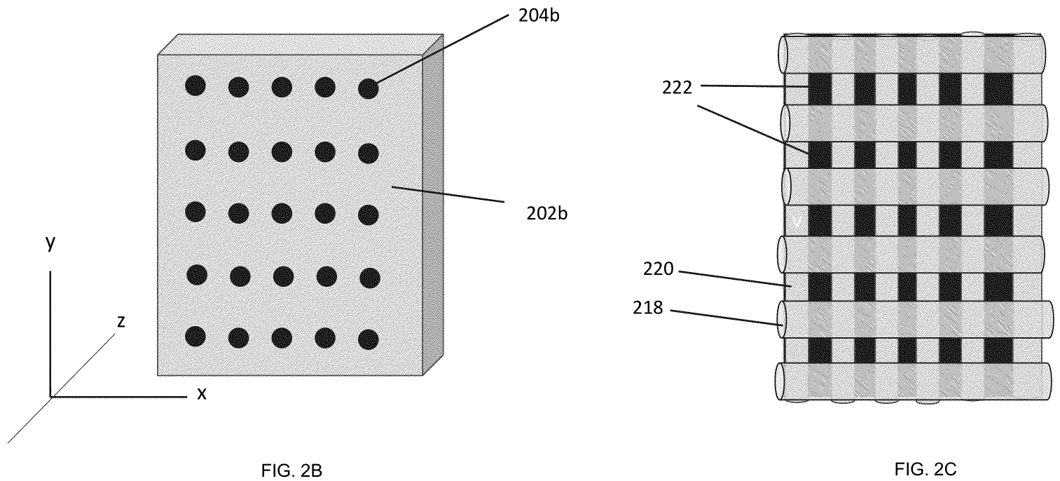

In a preferred embodiment, a stack of layers can be used to form an absorbent area, in which layers of absorbent material are separated by secondary absorbent sheets such as paper-based materials that are interposed therebetween. An illustrative embodiment of an absorbent area 200 according to these principles is depicted in FIG. 2A. Using this layered approach, the number of layers and their composition can be determined by the requirements of the particular useful article, considering factors such as the expected service duty, the liquid-holding capacity of the article and its component layers, and the desired overall thickness of the product. A multilayered structure, such as is depicted in FIG. 2A, comprises at least one primary absorbent layer and at least one secondary absorbent sheet; in embodiments, a plurality of absorbent layers can be separated by secondary absorbent sheets, as for example where at least one secondary absorbent sheet is interposed between two primary absorbent layers, which can be the same as or different from each other. A layer of absorbent material or a secondary absorbent sheet can optionally be embossed or imprinted or otherwise patterned to increase the effective surface area and/or to enhance lateral transmission of an incident fluid. In contrast to bulky conventional designs, such as debonded fluff pulp surrounding SAP bead cores, the layered design contemplated herein lends itself to a thin cross-sectional profile. The embodiment of a layered absorbent area 200 depicted in cross-section in FIG. 2A features layers of the absorbent material 202a as disclosed herein that can be separated by secondary absorbent sheets 208 to exploit the ability of the secondary absorbent sheets and any patterning added to their substances or their surfaces to increase effective surface area, to direct fluid flow or both, in combination with the ability of the layers of absorbent material 202a to absorb large amounts of fluid. The secondary absorbent sheets 208 can also have absorbent properties selected to improve the overall absorbency of the absorbent area 200. In embodiments, they can be formed of materials that are the same as or different in composition from the absorbent material 202 that forms the thicker layers. In preferred embodiments, the secondary absorbent sheets 208 are formed from a different material than the absorbent material 202a, for example, a paper-based material as has been described for use as a carrier material such as has been described in conjunction with FIG. 1. In a particularly preferred embodiment, the secondary absorbent sheets comprise or consist of paper-based materials that are highly wicking, low-density, high-porosity paper such as are used in filter paper, paper towels, tissue paper, or the like. Paper is a particularly advantageous material to place in layers above and below the layers of absorbent materials: because it is made of randomly stacked pulp fibers with voids in between, it acts as a co-continuous matrix, as described below in more detail. Desirably, a highly wicking, low-density, high-porosity paper-based material such as filter paper, paper towel, tissue paper, or the like, is employed rather than a coated paper or a dense sheet (e.g., office paper).The layered arrangement disclosed herein is consistent with a wide range of design choices and process latitude. For example, the top and bottom gel layers may have a different porosity from that of intervening layers, and the outside layers (top and bottom) can have different compositions and textures.

As shown in FIG. 2A, the layers of absorbent material 202a (i.e., primary absorbent layers) are provided with apertures, such as pores, holes, channels or other voids 204a that permit some of the fluid 214 encountering a layer to pass through it with minimal absorption. In other embodiments, the apertures such as pores, holes, channels or other voids can be designed to facilitate fluid flow in a particular direction, for example towards the center of the absorbent area, or to improve the overall absorbency of the layer. For example, certain of the pores extend transmurally, allowing flow-through passage of fluid, while other pores extend only partially into the layer, to act as fluid receptacles that encourage the fluid to be absorbed within that layer. Channels can also extend from peripheral to central to concentrate the fluid in a more absorbent central region, or from central to peripheral or otherwise to distribute a deposit of fluid more uniformly across the absorbent layer. While the layers of absorbent material 202a are shown as having a uniform thickness, it is understood that they can have any desired thickness and any sort of variation of thickness. For example, the primary layers of absorbent material 202a can be thicker in the middle of the absorbent area to improve absorption in that region, in the event that a larger volume of fluid is deposited there, or can be thicker along the edges to enhance absorption in those regions, in order to prevent leakage of fluid from the absorbent area 200 overall. Other arrangements of layer thickness can be engineered to accomplish specific goals of absorption. While spacing between layers can be provided in the absorbent area 200, more commonly the layers are juxtaposed closely to each other. In embodiments, the primary layers of absorbent material 202a and secondary absorbent sheets 208 are compressed together to produce a thinner cross-section for the absorbent article 200.

As further shown in FIG. 2A, optional specialized inner and outer layers can be added to the layered arrangements described above. For example, a top layer 212 of a softer material can be included as the point of contact with the skin or other sensitive surface. This top layer 212 can contain additives having advantageous properties, similar to those mentioned in conjunction with the top layer in FIG. 1: for example, additives such as plasticizers (e.g., glycerin, PEG, Pluronics, and the like) to impart product softness and superior hand-feel, or vitamins (e.g., Vitamin E) or medications to treat diaper rash or other skin conditions can be included in this inner layer. Desirably, the top layer 212 is permeable to the liquid 214 it encounters, so that the liquid 214 can be absorbed by the remainder of the absorbent area 200 without remaining in contact with the skin or other sensitive area. Whether to add a specialized top layer 212 to the absorbent area and the nature of such a specialized top layer 212 are design decisions that are based on the article of manufacture within which the absorbent area 200 is to be included. For example, a personal care item such as a diaper can have more use for a specialized top layer 212 close to the subject’s skin, in contrast to a pad that is designed for minimal body contact use such as dog training pads, or in contrast to a pad such as a disposable medical underpad in which skin contact does not exist or is not prolonged.

A bottom layer 210 can be optionally applied to the absorbent area 200 as well. In embodiments, the bottom layer has a barrier property: for example, it can be water-resistant or waterproof, and/or it can be oil and grease resistant. Water, oil, and grease resistance can be imparted by a number of techniques familiar in the art, depending on the need in a particular article of manufacture. For example, an outer waterproof layer for a personal care item can keep any absorbed fluid from passing out of the absorbent area 200 into the environment. Such a layer can also protect the absorbent area 200 from encounters with fluids in the environment. Desirably, any special properties that are imparted to the outer layer 210 are produced by using biopolymers, so that the entire article of manufacture is biodegradable.

FIG. 2B provides a top view of a primary layer of absorbent material 202b, corresponding to a similar layer (202a) shown in cross-section in FIG. 2A. As shown in FIG. 2B, a plurality of apertures 204b are arrayed symmetrically in an ordered geometric array, corresponding generally to the ordered geometric array shown in FIG. 2A. However, it is understood that the apertures can be arranged in any ordered pattern, in order to optimize drainage and/or absorbency of the layer, or the apertures can be arranged randomly. Optional indentations or channels (not shown) can be formed superficially in the absorbent material to direct fluid in the x-y plane without allowing the fluid to penetrate through the material in the z-plane. A layer of absorbent material 202b bearing an arrangement of apertures can be formed by methods familiar in the art. In an exemplary embodiment, such layers can be formed using industry-standard reel-to-reel processes, which is a well-established, economical manufacturing technique, especially when compared to air-laying. Recipe blending, followed by pouring the full formulation onto a moving silicone web (or other easy-to-release substrates, such as olefin or Teflon belts) can launch the process. The liquid used to form the absorbent layer can be optionally squeezed through a narrow gap under a roller to define the ultimate gel layer thickness. The continuous sheet then travels through a heating/drying tunnel or under a series of IR lamps. Many industrial drying options (e.g., microwave or vacuum-assisted evaporation) exist for this purpose. The dried sheet of absorbent material can be sandwiched between paper layers, and the process can then be repeated a designated number of times to produce the multi-layer stack. Those skilled in the art of reel-to-reel film fabrication and multi-layer stacking can easily develop numerous mechanical alternatives or adjuncts to enable the rapid and industrial-scale manufacture of articles incorporating the principles of the inventions disclosed herein. Once a continuous stack roll is manufactured, it can be cut into pieces matching the profile of an absorbent area for an article of manufacture such as a diaper or personal care item or other absorbent article. The newly created edges of the layered stack resulting from cutting (e.g., by punching with a sharp tool in one stroke) can be optionally pressed or otherwise adhered to each other to create strong cohesion, producing a multi-layered-stack-core that behaves like a monolithic integral article.

In embodiments, layers of absorbent material for use in forming absorbent areas for articles of manufacture can be molded or penetrated using conventional industrial techniques to create apertures, pores, channels or the like to regulate fluid flow, as described above. For example, the layer of absorbent material can be prepared as a sheet that is dried partially or completely, and is then punctured in order to create the apertures, pores or indentations in the surface. Alternatively, the absorbent material can be poured into a mold containing pore-forming elements and dried, so that when the material is removed from the mold it contains impressions, channels, or holes corresponding to the pore-forming elements.

A variety of methods for forming apertures, pores or indentations in sheets or layers of absorbent material are available besides penetration or molding. FIG. 2C depicts a top view of a primary layer of absorbent material that illustrates such an alternative embodiment, in which the apertures, pores or voids are formed by the overlay of overlapping strips, strands or blocks of the absorbent material. As shown in FIG. 2C, a primary absorbent layer of the multilayered structure in FIG. 2A can be formed from overlapping pieces of absorbent material that are arranged in a preselected design, for example a crisscross design. As shown in FIG. 2C, a bottom arrangement of vertically oriented pieces, strips or strands 220 of the absorbent material can be overlapped by a top layer of horizontally oriented pieces, strips or strands 218 of the absorbent material, or vice versa. Open areas 222 remain between the overlapping vertical and horizontal pieces, strips or strands, which allows these gaps in the engineered structure to act as pores that extend into or through the layer. The primary absorbent layer depicted in FIG. 2C can be separated by one or more secondary absorbent sheets to form an organized multilayered structure, such as is illustrated schematically in FIG. 2A. While FIG. 2C depicts a geometrically ordered arrangement of overlapping vertical and horizontal pieces, strips or strands, it is understood that any arrangement in which pieces of absorbent material overlap each other can produce apertures, pores, channels, or other such gaps that permit fluid to pass through the layer or to be trapped. For example, elongated strands of absorbent material can be piled on top of each other in an ordered or random manner, with apertures, pores or gaps thereby formed in between the strands. In embodiments, a primary absorbent layer can be entirely formed from overlapping pieces of absorbent material that produce gaps between them that permit fluid to pass into or through the layer, as depicted in FIG. 2C, while in other embodiments, such a layer can be formed that comprises regions or segments of overlapping pieces of absorbent material that produce gaps between them that permit fluid to pass into or through the layer; such regions or segments can be embedded in other materials that can be absorbent materials or that can be carrier materials as previously described.

Specialized foaming techniques and foamed products have been developed to form foamed absorbent materials that can be used as layers to form absorbent areas as disclosed herein. Two exemplary foam configurations have been identified that can be deployed to produce absorbent materials: (1) voids dispersed in a continuous gel layer, not extending too far as to form connectivity in-plane, i.e., x/y directions, but sufficient to lead to z-direction (i.e., thickness) penetration, thereby permitting with the spreading of the incident fluids to a subjacent layer, for example a paper layer or other absorptive layer, as described in more detail below; and (2) co-continuous, intertwined empty threads (e.g., elongate empty spaces) co-mingled with biopolymer threads constituting the porous gel matrix, in which case lateral and vertical spreading can both happen quickly within the gel layer. Control of foam morphology can take place by varying the surfactant concentration in the formulation, adjusting polymer concentration, and imposing a high degree of agitation prior to pouring the mixture onto a silicone substrate and drying to make the final foamy (i.e., porous) sheet. Foamed materials produced either in configuration (1) or (2) described above have advantageous properties for use as absorbent materials. For example, if the foam is produced with dispersed voids (as described above in configuration (1)), the incident liquid can wick down to the paper layer below and spread laterally quickly. This allows the layers of absorbent material above and below the paper layer to get exposed to the liquid efficiently, allowing it to be back-absorbed by previously unexposed regions of the gel layer.

In embodiments, formulations used for preparing foams as described above can comprise absorbent polymers, such as have been described herein. For example, a mixture of hydroxyethyl cellulose (HEC) and hydroxypropyl methylcellulose (HPMC) can be used in a ratio of 80:20 can be used, to form about 1.2% of a liquid formulation; the remainder of the liquid formulation is water, plus the plasticizer and surfactants that are added as described below. A plasticizer such as glycerol can be added to increase the flexibility of the final product, for example added to the absorbent polymer solution in a plasticizer:absorbent polymer ratio of about 5:95. Glucoside surfactants can be added to the plasticizer-polymer solution, in order to facilitate the foaming process for the solution. Useful surfactants include hexyl glucoside and capryl glucoside, which can be added together or individually. The surfactant or surfactants can be added in a surfactant:absorbent polymer ratio of about 1:1.5. Water is added to these ingredients to form the total volume of the formulation. The mixture thus produced is stirred to mix thoroughly, and then is whisked or otherwise agitated to produce a foamed consistency for the formulation. The foamed formulation can then be deployed to form a sheet, which can be dried in an oven to produce the final solid or semi-solid product that can be used to prepare absorbent areas for articles of manufacture, as described herein.

### 4. Additives to Absorbent Formulations

Additives having advantageous properties can be included in the high viscosity hydrogel-forming polymer and plasticizer mixture, and/or can be included in carrier layers in layered absorbent areas, such as have been described above with reference to FIG. 1, and in the layers of absorbent materials themselves, such as have been described above with reference to FIG. 2. Desirably, compostable and biodegradable materials from renewable sources can be used as additives. A wide range of additives can be included, whether as particles, fluids or emulsions, including without limitation activated charcoal, activated carbon, zeolites, bicarbonate powder, solid desiccant powder, emulsified oil and the like. Other desirable additives can be envisioned by those having ordinary skill in the art.

In more detail, as an example, materials such as activated charcoal, activated carbon, or biochar (made from coconut husk or other natural materials) and the like can be added as suspended particles in the hydrogel mixture to reduce odors and improve odor control, or they can be infused into one or more carrier layers, or both. Such materials are known to be porous and to trap toxic chemicals and odors. In embodiments, odor-absorbing or odor-neutralizing chemicals (e.g., beta-cyclodextrin, bicarbonate, pentane-1,5 diol, etc.), scents, fragrances, and other odorant modifiers can be advantageously introduced into the hydrogel mixture or embedded in the carrier material, or both. Essential oils and other fragrances can be provided in encapsulated form (e.g., encapsulated in cyclodextrins) for inclusion in the absorbent material, the carrier material, or both. Absorbent areas including odor-absorbent additives can be useful for personal care items such as diapers, incontinence pads, and feminine hygiene products. In other embodiments, absorbent areas including odor-absorbent additives can be useful for pet care products such as animal litter.

As another example, additives can be filler additives such as fluff pulp, microfibrillated cellulose, or nanofibrillated cellulose, which are materials that can decrease cost and/or to contribute to a softer or stable texture for the resulting absorbent material or carrier material or both. Softer, more conformable absorbent materials can be used for bandages or personal care items, where shape and malleability can improve performance. For pet care products, softening additives can improve “paw feel,” an important feature for products like animal litter. As another example, bicarbonate or starch can be added to improve dimensional stability. As yet another example, additives can be used that function as indicators. Color-changing indicators can be used to indicate when the absorbent material or carrier material or both is saturated, for example in a diaper or for animal litter. Indicators can also be used for diagnostic purposes, where the indicator reveals the presence of certain chemicals in the urine or certain pH changes in the absorbent material or carrier material or both. Such indicators can provide information about illness, disruption of homeostasis, etc., for veterinary purposes (in animal litter), or for pediatric or geriatric patients who are using the absorbent articles for personal care. Similarly, indicators in bandages comprising the absorbent materials disclosed herein can signal physiological changes when the absorbent material is exposed to body fluids.

Other additives having specialized properties can be included in the absorbent materials or carrier material or both as disclosed herein, to create a range of useful products via materials embedded in or attached to the matrix of the absorbent material or the matrix of the carrier material. For example, additives for personal care articles incorporated into the hydrogel itself or into the carrier material can include plasticizers (e.g., glycerin, PEG, Pluronics, and the like) to impart product softness and superior hand-feel. As another example, skin rejuvenating ingredients (e.g., hyaluronic acid, aloe vera, alpha-lipoic acid, and vitamins C&E) can be loaded in the hydrogel or embedded in the carrier material. Medication (e.g., hydrocortisone, anti-fungal agents) can be incorporated into the hydrogel or carrier material or both to produce vehicles for drug delivery, for example to treat diaper rash or other skin conditions. In other embodiments, wound dressings can be prepared using the absorbent materials and combinations thereof disclosed herein, with additional medications being included in the polymer layer or the carrier material layer or both, such as antiseptics, anti-microbial agents, blood clotting agents, and the like. In embodiments, the hydrogel layer either alone or in combination with carrier layers can be held in proximity to the skin or affixed thereto as a component of a bandage or dressing.

In embodiments, filler materials can be added to the hydrogel matrix to decrease material costs. For example, a polymer such as low molecular weight dextrin, dextran, or other low molecular weight carbohydrates can be added the high viscosity hydrogel-forming polymer and plasticizer mixture, which additives can also swell as the hydrogel forms. The swelling capacity of the added fillers can reduce the need for more expensive ingredients in the absorptive matrix.

While, in embodiments, fillers and smaller oligomers can be used as fillers to decrease cost, foaming can also be used to decrease cost and promote swelling. The formulation prepared as described above can be used, comprising the water swellable polymer and the plasticizer, but the mixture can be stirred such that bubbles are introduced to the viscous solution, thereby forming a foam. In other embodiments, surfactants (for example, non-ionic surfactants) can be used instead of or in addition to vigorous stirring to allow for bubble generation and subsequent foam formation. In embodiments, viscous solutions may promote foaming more easily, so ingredients known in the art to increase viscosity may be introduced into the mixture. The addition of foam (bubbles) allows for an increase in porosity of the hydrogel, thereby creating voids into which water can be imbibed and stored. This porosity within the hydrogel allows for the use of less intrinsically swellable polymer while still promoting comparable water entrapment. Other ingredients of the formulation as described above (e.g., crosslinkers) can be used in a similar manner if the hydrogel has been foamed.

High viscosity hydrogel-forming polymer and plasticizer mixtures as disclosed herein can be used as a coating for other particles or core materials to create composite absorbent materials. The coating mixture can include additives, examples of which are described above. In certain embodiments, a porous core material can be provided, to be enveloped partially or completely by the mixture. In other embodiments, the core material can be substantially solid and non-porous, to be enveloped partially or completely by the mixture. Desirably, the core material can be selected from renewable materials. In embodiments, the core material imparts advantageous properties such as stability to the coated composite structure, or improved odor uptake. Useful core materials include, without limitation, natural materials such as activated carbon, charcoal, biochar, walnut or other nut shells, sawdust, fluff pulp, corn husk, psyllium husk, and the like. In embodiments, the core material would be between about 0.2 mm and about 5 mm in size, or between about 0.2 and about 2 mm in size. In embodiments, the coat thickness would be between about 1 micron and about 100 microns in thickness, applied either uniformly or non-uniformly over the surface of the core material. In embodiments, coated particles could be produced by adding the core particles and the high viscosity hydrogel-forming mixture in a mixing tank, and heating the resulting combination with heating elements or other similar equipment (oven, fluid bed dryer, etc.). During or after heating, the core particles are stirred or agitated in the mixture to separate them and allow for even coating (not necessary if a fluid bed dryer is used).

### 5. Exemplary Articles of Manufacture

### a. Personal Care Items

I A variety of personal care articles can be formed using the swellable materials and absorbent areas as disclosed herein, either separately or combined with other, non-swellable materials in arranged structures such as the absorbent areas that have been described with reference to FIG. 1. In embodiments, absorbent articles of manufacture such as diapers, incontinence pads, feminine hygiene products, and the like, can be formed more economically, and worn more conveniently and comfortably, with retention of performance attributes such as moisture wicking and breathability by incorporating absorbent areas such as are described herein.

In embodiments, use of the swellable materials and the use of absorbent areas formed from such swellable materials, all as disclosed herein, can improve the biodegradability or compostability of a disposable personal care item such as a baby diaper or adult incontinence pad. As an example, a conventional disposable diaper includes the following components: a top sheet or inner layer that contacts the baby’s skin and forms the first point of contact for waste fluids; an absorbent core area that absorbs and retains the waste fluids; and a waterproof back sheet or outer layer that provides a barrier that keeps the fluids inside the diaper and provides leak protection. The top sheet in conventional disposable diapers is commonly made from woven, non-woven, or porous formed-film polyethylene or polypropylene materials. The back sheet can be formed from the same material as the top sheet, but typically includes an inner film barrier that keeps fluids inside the diaper along with an outer surface that is soft to the touch. SAPs for fluid absorption are disposed in the absorbent core. If the absorbent materials and absorbent areas formed therefrom, all as disclosed herein, replace traditional, petroleum-derived SAPs, an element impairing biodegradability and compostability is removed from the product: it therefore becomes more sustainable, while still retaining the desirable performance features of the conventional version. Improving sustainability further, the petroleum-derived materials used for the top sheet and the back sheet can be replaced with bio-based materials that are themselves biodegradable and compostable as described herein, aiming for a fully biodegradable and/or compostable disposable diaper. Absorbent areas as disclosed herein can be incorporated within traditional diaper construction to produce an article of manufacture that is fully biodegradable and/or compostable. Moreover, absorbent materials and absorbent areas formed therefrom, all as disclosed herein, can be incorporated into a disposable structure that can be used in other personal care products besides disposable baby diapers.

In an embodiment, an article of manufacture such as a traditional diaper or personal care product construction can be modified to include a reusable external covering and a disposable internal component comprising the absorbent area. In an embodiment, this construction comprises a reusable outer shell such as a (semi-permanent) elastic mesh and a (used-once) disposable integrated absorbent structure or absorbent area formed from bio-based absorbent materials, as disclosed herein. The elastic mesh is used to position the disposable absorbent interior structure in proximity to an anatomically advantageous area within the article of manufacture so that the disposable absorbent interior structure can wick the body fluids away from their point of delivery and absorb them. An anatomically advantageous area is a region of the article of manufacture that is expected to encounter the body fluids to be absorbed, such as urine or feces for personal care items, menstrual flow for sanitary napkins, wound drainage for medical or surgical dressings, and the like. The absorbent interior structure, besides being removable and disposable, permits the sanitary and efficient collection of body fluids so that they can be readily disposed of during the removal of the absorbent inner structure. When it comes time to change the core, the caretaker simply pulls open the shell, exposing the core. Since the core is a thin-profiled, paper-gel stack, the whole article can be rolled up into a wrap and disposed into a compost pile or garbage bin. Optionally, the disposable absorbent interior structure can be affixed to the reusable external covering with a biodegradable adhesive, which will allow the interior structure to be lightly anchored to the reusable exterior structure.

In embodiments, disposable absorbent interior structures are formed comprising at least the following layers: (a) a layer closest to the skin, a skin-protective layer that wicks body fluids towards the inner absorbent core; (b) external to that layer, an absorbent area comprising the absorbent materials as described herein, for example disposed within a matrix formed from biobased materials (wood pulp, fluff pulp, microfibrillated cellulose, or the like) or disposed as components within an organized multilayered structure as described above; and (c) external to that layer, a hydrophobic layer such as a hydrophobically treated paper layer that retains the moisture in the absorbent core area and prevents leakage. This absorbent structure comprising (i) an inner skin-protective layer, (ii) one or more absorbent layers comprising absorbent materials and optionally carrier materials (the absorbent core), and (iii) an outer hydrophobic layer, can be constructed as one single structure that includes layers for all three properties (such as is depicted in FIG. 1), or as a plurality of layers that among them provide an inner skin-protective layer, an absorbent core, and an outer hydrophobic layer.

In embodiments, an article comprising a reusable shell and an absorbent core as described herein can be formed with a very thin overall profile. Disposable absorbent interior structures consistent with the principles of the invention can be formed so that they can be changed/replaced when soiled, without having to change/replace the reusable mesh layer, which remains in place. Advantageously, the disposable core is thin and lightweight, convenient and comfortable to wear, for example under clothing for an adult incontinence pad. Moreover, the small size and low weight of the product makes it suitable for business models such as a subscription-type home delivery, enhancing convenient use.

The reusable exterior covering can be made of materials such as stain-repellent cotton, cotton mix-woven with Lycra (stretchy cotton), polyester/cotton blend, athletic-wear-grade nylon, and the like, which will permit the covering to be laundered and used for prolonged periods of time. The external mesh can be constructed to conform to the wearer’s body contours, and can be made of durable elastic material (e.g., Lycra, Spandex, silicone, etc.). This mesh architecture for the external covering is porous, thus highly breathable. Besides the convenience of this approach, it can reduce skin contact time with excretions, and mitigate resulting skin irritation problems like diaper rash. In embodiments, fasteners can be metallic snaps, which are robust, easy to use, and engineered for repeated uses. The design style can be colorful and even personal. The leg holes can have high-quality elastic bands for form fitting. To open the shell, one can simply un-snap the metallic snaps and fully open the shell to allow retrieval of the spent/soiled core and reapply a new core. Other modifications consistent with the purpose of the reusable exterior covering and with the commercial direction of the product can be readily envisioned by skilled artisans in the field.

Advantageously, the absorbent core formed according to these systems and methods consists of naturally derived, biodegradable materials that are able to deliver exceptional performance. In embodiments, the performance of the absorbent core materials is comparable or superior to that of most conventional disposable diapers, whose core is bulky and made of synthetic, non-degradable SAP (super-absorbent polymer) beads and expensive, hammermill-debonded fluff pulp. Moreover, while conventional disposable absorbent materials such as are used in diapers and other personal care products are typically manufactured by both capital and process intensive air-laying, the innovative core disclosed here is manufactured using well-established, rapid, continuous, reel-to-reel process, significantly lowering the cost of goods.

The core-shell diaper construction, made feasible by our development of proprietary, biodegradable, high-absorbency, fast-wicking, paper/gel multi-layer stack, has important implications for ecology and sustainability. Not only is petroleum consumption (necessary for making synthetic SAP beads) substantially reduced or even eliminated, but also the thin core is entirely compostable. Since the shell is reusable, this invention has the potential to conserve a significant amount (30% by some estimates) of future landfill volume, assuming that traditional disposable diapers are substantially replaced by this new technology.

Additional features can be introduced to the core-shell assembly. For example, the core may have raised “dams” around the edges, e.g., by folding the edges before pressing (as stated in a previous section) to make the edges thicker to help contain the waste. Numerous geometrical and design features may be envisioned and incorporated to promote core-shell integration. All such refinements shall fall within the spirit and scope of the present invention.

In embodiments, many design refinements and functional additives may be incorporated and built upon this foundational invention. For example, functional additives such as odor absorbers may be integrated into the gel layer or sprinkled between layers. Or, for example, functional additives containing natural ingredients such as pulverized activated carbon (i.e., bio-char, made from pyrolysis of agricultural residues), beta-cyclodextrin, and chitosan biopolymer can be used. Similarly, functional additives such as fragrances (e.g., citrous oil (d-limonene), lavender oil, and numerous other essential oils for aromatherapy) can be included. In embodiments, functional additives such as antimicrobial and anti-fungal ingredients may be optionally added to the gel. The top-most paper layer may include health and wellness sensors, such as pH, glucose dehydrogenase (diabetes detection), protein colorimetric assays (to detect early-onset kidney malfunction), among many other urine-based lab-test chemistries.

This approach to diaper and personal care product construction, using replaceable absorbent inserts analogous to changeable filters for coffee machines or vacuum cleaners, is already familiar to the consumer, and offers distinct price and environmental advantages since only a small portion of the overall article requires disposal. By using biodegradable materials in the absorptive core instead of traditional synthetic SAPs, the absorbent material is intrinsically less environmentally burdensome. By providing a smaller sized absorbent core for disposal, as compared to the traditional form factor for a disposable diaper, the absorbent personal care product is suitable for small-scale composting instead of large-scale disposal facilities. Overall, this approach can enable environmentally sustainable reimagination of the whole disposable diaper industry.

### b. Animal Litter

Materials used for traditional animal litter (e.g., cat litter or “kitty litter”) are typically not derived from renewable resources. Clay materials predominate, in particular bentonites such as sodium bentonite or calcium bentonite. Other clays added to animal litter mixtures are sepiolite, montmorillonite, and kaolinite, depending on whether the formula is intended to clump on exposure to cat urine or not. Such materials are hydrous aluminum silicates, in which the trapped moisture creates a negative overall ionic charge that attracts water and body fluids, resulting in fluid uptake and swelling of the material. In certain products, sodium silicate crystals are used in addition to or instead of clays. All such materials, however, are not biodegradable or compostable. After use, animal litter materials are usually discarded as municipal solid waste, having a lifespan of millennia when relegated to landfills.

Desirable properties for animal litter include biodegradability, high rate of absorption and absorption volume capacity, cohesion, clumpability, ammonia and other odor masking, a density and texture that is acceptable for the target animal, clump strength, tendency to remain clumped, clump weight, and cost. Substituting absorbent hydrogel materials as disclosed herein for conventional animal litter sorbents can produce a biodegradable animal litter that imposes less of a burden on the environment. Biodegradable cat litter can be produced from absorbent hydrogel materials as disclosed herein that include odor absorbent additives, in combination with other bio-based materials that improve the texture, strength, dust protection, and/or cost of the final product.

### c. Medical Uses

The absorbent materials disclosed herein can be adapted for a number of medical uses, such as wound treatment, blood coagulation, treatment of a skin condition, surface application of a medical or wellness treatment, and transdermal dissemination of a pharmaceutical treatment. These articles can be used as carriers for other active ingredients, such as pharmaceutical products, wellness agents, vitamins, nutrients, and the like, to bring those active ingredients into contact with areas of the body that can benefit from exposure thereto.

### EXAMPLES

Materials and equipment used in Examples 1-5 include:
Corning stir plate
BINDER forced convection oven
Sigma Aldrich Chemicals
   Guar Gum
   Pectin
   Dextrin
   Alginate
   Locust Bean Gum
   Glycerol
   Hydroxyethyl Cellulose [HEC]
   Sodium Chloride
   Calcium Chloride
   Ammonium Chloride
   Magnesium Sulfate
   Sodium Sulfate
   Sodium Carboxymethylcellulose
Other Chemicals:
   Xanthan Gum: Amazon
   Super Absorbent Beads: Amazon
   FILMKOTE 54: Ingredion
   ERYSIS GE-36: Huntsman

### Example 1: Unary Biodegradable Absorbents

In this Example, various natural polymers and compounds (listed in Table 2 below) were prepared in aqueous solutions at 5 wt.% concentrations. Each solution was deposited into multiple wells on a hemispherical silicone mold (1 cm diameter) and dried in a BINDER forced convection oven at 70° C. For the first set of tests, the resulting solid dry gels were tested for swelling capacity by placing them in a small metal mesh cage that was subsequently submerged in a 0.104 M simulated urine solution (prepared in accordance with Table 1 below) at 25° C. for one minute. The mesh cage was then retracted from the urine solution, excess water was allowed to drip out for one minute, and paper towel was used to wipe off residual moisture around the cage before measuring the weight of the swollen gels. For the second set of tests, the experiment was reproduced under the same conditions (0.104 M urine solution at 25° C.) but the mesh cage containing the dry solid gel polymer/compound was submerged for 10 minutes to assess if the natural polymers/compounds continued to swell with time. Absorption capacity for each sample was calculated according to EQ1 below and resulting data is tabulated for the one- and ten-minute immersion experiments in Tables 2 and 3, respectively.

$$E Q 1 : A b s o r p t i o n C a p a c i t y = \frac{\left(w e t w e i g h t - d r y w e i g h t\right)}{d r y w e i g h t}$$

TABLE 1

| | |
|---|---|
| Salt | Concentration (mol/L) |
| Calcium Chloride | 2.59 × 10⁻³ |
| Sodium Chloride | 0.089 |
| Magnesium Chloride | 2.49 × 10⁻³ |

TABLE 2

| Polymer/Compound | Absorption Capacity |
|---|---|
| SAP | 10.9 |
| Xanthan Gum | 4.43 |
| Guar Gum | 3.15 |
| Pectin | 2.12 |
| FILMKOTE 54 | 3.89 |
| Alginate | 4.91 |
| Locust Bean Gum | 5.08 |

TABLE 3

| Polymer/Compound | Absorption Capacity |
|---|---|
| Xanthan Gum | 8.83 |
| Pectin | 6.09 |
| FILMKOTE 54 | 6.13 |
| Alginate | 9.17 |
| Locust Bean Gum | 5.27 |

### Example 2: Binary Biodegradable Absorbents

Samples for this Example were prepared by dissolving glycerol in DI water, followed by adding a natural polymer or compound, as listed in Table 4 at 2.5 wt.%. In each solution, glycerol was added at 10 wt.% of the natural polymer/compound weight. Drying protocols delineated in Example 1 were followed for the samples prepared from the polymers/compounds listed in Table 4, and the dried samples were tested using Example 1’s one-minute immersion protocol, except that the temperature of the simulated urine was 37° C. Results are summarized in Table 4 below.

TABLE 4

| Polymer/Compound | Absorption Capacity |
|---|---|
| Alginate | 12.1 |
| Guar Gum | 4.20 |
| Hydroxyethyl Cellulose | 10.3 |
| Locust Bean Gum | 5.25 |
| FILMKOTE 54 | 6.85 |
| Xanthan Gum | 6.69 |

### Example 3: Tertiary Biodegradable Absorbents

In this Example, tertiary systems were prepared by dissolving glycerol in DI water, followed by adding mixtures of two natural polymers in the ratios set forth in Table 5, for a combined concentration of 2.5 wt.%. Glycerol was added at 30 wt.% of the combined natural ingredients. For example, a 300 g sample solution contained 290.25 g water, 2.25 g glycerol, 6.75 g HEC, and 0.75 g alginate.

Drying protocols delineated in Example 1 were followed for the samples prepared from the polymer pairs listed in Table 5, and the dried samples were tested using Example 1’s one-minute immersion protocol, except that the temperature of the simulated urine was 37° C. The absorption capacity for each pair of polymers is listed in Table 5 below.

TABLE 5

| Polymers | Component Ratio | Absorption Capacity |
|---|---|---|
| Hydroxyethyl Cellulose + Alginate | 9:1 | 10.7 |
| Hydroxyethyl Cellulose + Xanthan Gum | 9:1 | 7.41 |

### Example 4: Varying Molecular Weight of Natural Absorbent Polymer

Four different molecular weights of hydroxyethyl cellulose (listed in Table 7) were prepared as 1 wt% aqueous solutions. Glycerol was also included in each solution at 10wt% of the polymer mass. Samples were dried with the same procedures as Example 1 and then were tested in the 0.154 M simulated urine aqueous solution set forth in Table 6. Drying protocols delineated in Example 1 were followed for the samples prepared from the four molecular weights of hydroxyethyl cellulose listed in Table 7, and the dried samples were tested using Example 1’s one-minute immersion protocol with the simulated urine of Table 6, with the temperature of the simulated urine at 37° C. The calculated absorption capacity for each sample is set forth in Table 7.

TABLE 6

| Salt | Concentration (mol/L) |
|---|---|
| Calcium Chloride | 2.59 × 10⁻³ |
| Sodium Chloride | 0.089 |
| Magnesium Sulfate | 1.97 × 10⁻³ |
| Sodium Sulfate | 8.36 × 10⁻³ |
| Ammonium Chloride | 0.024 |

TABLE 7

| Hydroxyethyl Cellulose Molecular Weight | Absorption Capacity |
|---|---|
| 90 KDa | 11.1 |
| 380 KDa | 5.89 |
| 720 KDa | 13.5 |
| 1.3 MDa | 20.0 |

### Example 5: Covalently Crosslinked Natural Absorbent Polymer

This Example tests a tertiary composition of hydroxyethyl cellulose, alginate, and glycerol, prepared as described in Example 3, in both a non-crosslinked form and with crosslinking. For the crosslinked form, a crosslinker (triglycidyl ether of propoxylated glycerin) and catalyst (butyltriethylammonium chloride) were mixed into the hydroxyethyl cellulose/alginate/glycerol solution at 1 wt% of the combined weight of HEC and alginate. As an example, a solution of hydroxyethyl cellulose, glycerol, alginate and crosslinker can include the following ingredients: 290.10 g DI water + 2.25 g glycerol + 6.75 g HEC + 0.75 g alginate + 0.075 g crosslinker + 0.075 g catalyst. The resulting amalgam was dried according to the drying protocol of Example 1, and each was tested using Example 1’s one-minute immersion protocol, except that the temperature of the simulated urine was 37° C. The absorption capacity of both the crosslinked and the non-crosslinked samples are listed in Table 8 below.

TABLE 8

| Polymer(s)/Compound(s) | Crosslinking | Component Ratio | Absorption Capacity |
|---|---|---|---|
| Hydroxyethyl Cellulose + Alginate | N/A | 9:1 | 10.7 |
| Hydroxyethyl Cellulose + Alginate | Erisys GE-36 | 9:1 | 12.9 |

Materials and equipment used in Examples 6-7 include:
Corning stir plate
BINDER forced convection oven
Mesh tea filter
Syringe
Syringe Needles
Beakers
Deionized (DI) Water
Sigma Aldrich Chemicals
   Gelatin
   Sodium Alginate (Alginate)
   Guar
   Dextrin
   Sodium Chloride
   Calcium Chloride
   Boric Acid
   Sodium Tetraborate
   Glycerol
Other Chemicals:
   Xanthan Gum (XG): Amazon
   Super Absorbent Beads: Amazon (ASIN B075DX8PZ2)
   Erisys GE-36

### Example 6: Swellable Beads

This experiment was performed to create hydrogel beads made of natural materials, to test bead formation, and to measure the extent to which they would swell when exposed to DI water.

A viscous solution of hydrogel polymer (as specified in Table 9) was made by weighing out certain ratios (all specified in Table 9) of polymer powders or single polymer powders (if a non-mixture was used) and slowly adding the powders to DI water while the mixture was stirred on a stir plate, initially with aggressive stirring for 15-20 minutes, and then on a slower speed until all powder was dissolved and the solution was homogeneous. Separately, 40 g of crosslinking solution was made (varied for each test, as specified in Table 1) by adding the crosslinker in powder form to DI water and mixing until all powder was dissolved. The crosslinking solution was stirred gently on a stir plate in a beaker while about 3ml of each viscous hydrogel solution (prepared as previously described) was added into it dropwise. It was observed that the added hydrogel formed into beads almost immediately upon being dropped into the CaCl2 crosslinking solution, while other crosslinking solutions did not help to form beads as well. The hydrogel beads that formed in the crosslinker solution were filtered over a mesh screen (if possible), washed thoroughly with DI water, and dried overnight in an oven at 60° C.

Hydrogel beads were formed using the procedure above with a variety of hydrogel components, hydrogel concentrations, hydrogel ratios, and crosslinking techniques, as set forth in Table 9. These sample beads were then tested in accordance with the following protocol:

Before testing, all samples were placed back into the oven at 60° C. for 15 minutes to ensure that no moisture was present in the beads. For each test, 30 g of testing solution (as specified in Table 2, either DI water or a 0.9% NaCl solution at room temperature) was weighed out into a beaker, and roughly 0.2 g of beads from each tested sample were weighed out in a mesh tea filter. The mesh tea filter containing the beads was immersed in the beaker containing the testing solution and swirled gently (X-Y direction only) for one minute. After one minute, the mesh tea filter was removed and allowed to drain. The bottom of the filter was wiped with a paper towel, and the beads were weighed again to determine liquid uptake and overall swelling. For a control, commercially available SAP beads were tested following the same protocol. Results for the control tests and the sample tests, plus observer comments, are set forth as seen in Table 10.

TABLE 9

| Sample | Hydrogel Solution Concentration | Hydrogel Mixture | Hydrogel Ratio | Crosslinker | Crosslinker Solution Concentration |
|---|---|---|---|---|---|
| 1 | 10% | Gelatin/Alginate | 50/50 | CaCl2 | 50% |
| 2 | 10% | XG/Alginate | 50/50 | CaCl2 | 50% |
| 3 | 10% | XG/Alginate | 80/20 | CaCl2 | 6.7% |
| 4 | 10% | XG/Alginate | 80/20 | CaCl2 | 50% |
| 5 | 10% | XG | N/A | CaCl2 | 50% |
| 6 | 1% | Guar | N/A | Boric Acid | 3% |
| 7 | 1% | Guar | N/A | Sodium Tetraborate | 3% |
| 8 | 10% | XG/Alginate | 80/20 | CaCl2 | 1% |
| 9 | 10% | XG/Alginate | 80/20 | CaCl2 | 3% |
| 10 | 10% | XG/Alginate | 80/20 | CaCl2 | 5% |
| 11 | 1% | XG/Alginate | 90/10 | CaCl2 | 1% |
| 12* | 5% | Alginate | N/A | CaCl2 | 1% |
| 13* | 2.5% | Alginate | N/A | CaCl2 | 1% |
| 14* | 5% | Alginate/Dextrin | 50/50 | CaCl2 | 1% |

*Represents samples where a 20 g bath of crosslinker was used, rather than a 40 g bath

TABLE 10

| Sample | Testing Solution | Weight Gain (%) | Observations |
|---|---|---|---|
| Control - SAP | DI | 6873 | |
| Control - SAP | DI | 7135 | |
| Control - SAP | 0.9% NaCl | 1318 | |
| Control - SAP | 0.9% NaCl | 986 | |
| 1 | DI | 60 | Well-formed round beads formed |
| 2 | DI | 65 | Well-formed round beads formed – teardrop shape due to higher viscosity |
| 3 | DI | 111 | Well-formed round beads formed – beads were not as hard – more “squishy” |
| 4 | DI | 52 | Well-formed round beads formed |
| 5 | DI | N/A | Beads were not solid enough to form or test |
| 6 | DI | N/A | Beads were low density and not viscous. Beads were not solid enough to form or test. |
| 7 | DI | N/A | Beads were low density and not viscous. Beads were not solid enough to form or test. |
| 8 | DI | 171 | Well-formed round beads formed |
| 9 | DI | 210 | Well-formed round beads formed |
| 10 | DI | 133 | Well-formed round beads formed |
| 11 | DI | N/A | Beads were not solid enough to form or test |
| 12 | 0.9% NaCl | 217 | Well-formed round beads formed |
| 13 | 0.9% NaCl | 113 | Well-formed round beads formed |
| 14 | 0.9% NaCl | 93 | Well-formed round beads formed - milky white color |

### Example 7: Extruded Strands

A 10% mixture of hydroxyethyl cellulose and sodium alginate (80/20 ratio) can be made up in a mixing tank by slowly adding the polymer powder into a 10% glycerol in DI water solution and aggressively stirring for 20 minutes, and then slow stirring until all polymer is dissolved, to produce a thick, viscous solution. Erisys GE36 multifunctional epoxy crosslinker can then be added at 5% the weight of the total polymer addition. The hydrogel mixture with added crosslinker can be put into an extruder with devolatilization capabilities, and then can be extruded (with much of the water evaporated from the heating process within the extruder) out as a strand of dewatered, hardened polymer, with a strand diameter of around 1 cm. This strand can be transferred to a conveyer oven where it can be heated at 60° C. for 2 hours. Upon removal from the oven, the strands can be cut into cylindrical small pellets (1 cm in length) to form dried beads, or they can be cut into long strands, about 3 inches in length and about 1 cm in diameter. Other shapes and diameter to length ratios can be prepared as well, using similar techniques.

The pellets and strands can be tested in 0.9% NaCl solution at body temperature to test swelling capacity. Possible swelling capacity results for beads of about 1 cm in diameter can include weight gain of about 1400-1600%. Possible swelling capacity for strands of about 3 inches in length can include weight gain of about 1200-1400%.

Materials and equipment used in Examples 8, 9, and 10 include:
Stir plate
VWR forced convection oven
OniLAB overhead stirrer
Sigma Aldrich Chemicals
   Hydroxy ethylcellulose (HEC)
   (Hydroxypropyl) methylcellulose (HPMC), Mn~ 120,000, and Mn~ 10,000
   Glycerol
   Capryl Glucoside
ThermoFischer extruder (single or parallel screw)
Slot- Die extruder
Doctor blade
Supports
   Flat Silicone sheet
   Flat carbon steel pan
   Silicone molds with 200+ cavities

### Example 8: Solutions for Sheets

In this example, a solution having absorbent properties was made and then was spread out into a sheet. The absorbent polymers (HEC and HPMC) were summed together at a concentration of 1.2% of the total solution. The ratio of absorbents to glycerol was equivalent to 95:5, and the ratio of absorbents to surfactant (capryl glucoside) was 1.5:1, with water making up the remaining amount of the solution. To prepare the solution, water was added to a beaker followed by glycerol. This beaker was placed on the magnetic stir bar and stirred for about 5 minutes with medium shear. Next, the surfactant was added to the beaker, and the beaker was placed on the overhead stir bar at 150 rpm. The absorbents are then weighed out and added to the beaker as the rpm was increased to 300. This solution mixes for 5-10 minutes at 300 rpm, then the mixing speed was brought back to 150 rpm and left overnight to stir (about 12 – 24 hours). Once the solution was fully homogenized, a doctor blade was used to spread the solution evenly on either a carbon steel pan or a silicone mat as a support. The thickness of the sheet was prepared at a thickness of 2 mm, although it is recognized that the thickness can be anywhere between about 0.5 mm through about 4 mm. These sheets are placed in the oven at 70° C. and left to dry.

Once dry, the sheets were peeled off the support and cut into rectangular pieces for absorption testing. For this test, a 0.9% NaCl in water bath was prepared, to simulate the composition of urine. 425-micron sieves are added to the bath so that the water reaches about halfway up the top opening. Each sample of the sheet was weighed dry then placed into the saline bath and left for about 1 minute. These pieces are then taken out of the water and weighed to determine the expansion of the sample. Expansion was determined by dividing the dry weight from the wet weight. Expansion data is listed in Table 11 below.

TABLE 11

| Trial | Polymer Used | Support | Dry Weight | Wet Weight | Expansion |
|---|---|---|---|---|---|
| 1 | HEC, HPMC Mn~120,000 | Carbon Pan | 0.06 g | 1.11 g | 18.5x |
| 2 | HEC, HPMC Mn~120,000 | Silicone Mat | 0.05 g | 1.03 g | 20.6x |
| 3 | HEC, HPMC Mn~ 10,000 | Carbon Pan | 0.04 g | 0.82 g | 20.5x |
| 4 | HEC, HPMC Mn~10,000 | Silicone Mat | 0.07 g | 1.34 g | 19.1x |

### Example 9: Solutions Plus Carriers

In this example the solution was used to dispense on a substrate. Following the same protocol from Example 8, the polymer solution was made. The polymer solution was added to an extruder/ slot die to dispense long strands of the solution onto a paper towel, tissue paper, or fluff pulp substrate. Most of the water evaporated during the extrusion heating process, leaving a hardened polymer on the substrate. Next, this sample was placed in the oven at 70° C. and left until fully dry. These samples were tested in 0.9% NaCl solution bath for absorption as was tested in Example 8 above.

### Example 10: Polymer Network

The solution prepared according to Example 8 was used to create a polymer network. After the solution had been prepared, it was dried on a silicone baking mold with many cavities, which allowed the solution to become embedded within the crevices and cavities in the x and y direction to produce a preselected network-like conformation. After drying, the mold was flipped upside down and the dried material was expelled with its conformational features preserved. The shaped material with its conformational features was then placed in the oven at 70° C. and dried further. After sufficient drying, the entire shaped structure was placed within a 0.9% NaCl bath with a strainer/ sieve underneath it; it was left in the bath for one minute and then removed. The expansion was then recorded by dividing the dry weight from the wet weight. Multiple shaped structures can be layered with paper towel, tissue paper, napkins, or fluff pulp, and such shaped structures can have different shape configurations in order to provide different mechanical or absorptive properties to a specified layer, and/or to promote passage of fluids into or through the layer at different rates. Expansion data is listed within Table 3, the high MW HPMC polymer was used for these tests.

TABLE 12

| % Absorbents | Trial | Dry Weight (g) | Wet Weight (g) | Expansion | Average |
|---|---|---|---|---|---|
| 3% | 1 | 0.67 | 7.92 | 11.821x | 14.414 |
| 3% | 2 | 0.43 | 7.39 | 17.186x | |
| 3% | 3 | 0.51 | 7.26 | 14.235x | |
| 4% | 4 | 1.56 | 9.87 | 6.327x | 7.184 |
| 4% | 5 | 1.11 | 8.74 | 7.874x | |
| 4% | 6 | 1.34 | 9.85 | 7.351x | |

### Example 11: Formulations for Absorbent Sheets

In this example, HEC made up 0.5 to 10% of the total solution, with glycerol added in a ratio of 95:5 of HEC to glycerol, and surfactant added at a 1.5:1 ratio of HEC to capryl glucoside, with the rest made up of water. First the glycerol was added to the water and placed on a magnetic stir bar for 5 minutes. Next capryl glucoside was added to the glycerol water mixture and moved over to the overhead mixer. Lastly, HEC was weighed out and added to the mixture slowly. The overhead mixer’s rpms was increased to 300 and held at that level for 5-20 minutes. The speed was then reduced to 150 rpm and the solution was stirred overnight at that speed to homogenize. This solution was then spread out on a silicone mat or a carbon metal pan with a doctor blade to a uniform thickness of 1.5 mm, and placed in the oven at 45° C. for 5 hours. When the sheets were taken out of the oven, they were peeled off the substrate and cut into small rectangles for absorption testing. The test method explained in Example 8 was performed to determine how much the sheets expand due to water. Expansion data is listed in Table 13 below.

TABLE 13

| % HEC | Dry Weight (g) | Wet Weight (g) | Expansion |
|---|---|---|---|
| 0.5% | 0.04 | 0.43 | 9.750 |
| 1.2% | 0.06 | 1.08 | 17.000 |
| 2.5% | 0.17 | 2.17 | 11.765 |
| 5.0% | 0.35 | 0.60 | 0.714 |
| 10.0% | 0.82 | 1.32 | 0.610 |

### Example 12: Comparing HEC:HPMC Ratios

Experiments were performed to optimize the ratio between HEC and HPMC in the recipe. Solutions were prepared as they were in Example 8 with ratios of 95:5, 90:10, 80:20, 70:30, and 60:40 ratios of HEC:HPMC. These solutions were dried on silicone mats or carbon metal pans and tested for expansion using the same methods as stated in Example 8 above. Expansion data are listed in Table 14 below.

TABLE 14

| Ratio of HEC:HPMC | Support | Dry (g) | Wet (g) | Expansion |
|---|---|---|---|---|
| 95:5 | Silicone Mat | 0.06 | 0.89 | 14.833 |
| 95:5 | Carbon Pan | 0.13 | 2.13 | 16.385 |
| 90:10 | Silicone Mat | 0.13 | 2.66 | 20.462 |
| 90:10 | Carbon Pan | 0.21 | 4.18 | 19.905 |
| 80:20 | Silicone Mat | 0.07 | 1.71 | 24.43 |
| 80:20 | Carbon Pan | 0.07 | 1.60 | 22.86 |
| 70:30 | Silicone Mat | 0.06 | 0.88 | 14.667 |
| 70:30 | Carbon Pan | 0.15 | 2.15 | 14.333 |
| 60:40 | Silicone Mat | 0.09 | 1.09 | 12.111 |
| 60:40 | Carbon Pan | 0.21 | 2.83 | 13.467 |

### Example 13: Foamed Sheets

In this Example, the solution was prepared as described in Example 8. Once the solution was homogenous it was poured into a small mixing bowl and beaten with a whisk to create bubbles. This solution, with the new bubbles, was then poured out onto a silicone mat, dried, and tested for expansion as described in Example 8. Expansion data for foamed sheets are listed in Table 15.

TABLE 15

| Thickness (mm) | Trial | Dry (g) | Wet (g) | Expansion | Average |
|---|---|---|---|---|---|
| 1.5 | 1 | 0.21 | 4.62 | 22.000 | 23.360 |
| 1.5 | 2 | 0.26 | 6.17 | 23.73 1 | |
| 1.5 | 3 | 0.20 | 4.87 | 24.350 | |
| 2.0 | 4 | 0.26 | 5.06 | 19.462 | 19.205 |
| 2.0 | 5 | 0.23 | 3.83 | 16.652 | |
| 2.0 | 6 | 0.18 | 3.87 | 21.5 | |

While this invention has been particularly shown and described with references to preferred embodiments thereof, it will be understood by those skilled in the art that various changes in form and details may be made therein without departing from the scope of the invention encompassed by the appended claims.

1. An absorbent material, comprising:
at least one bio-based hydrogel-forming swellable polymer; and
a plasticizer;
wherein the absorbent material is biodegradable, and
wherein the absorbent material demonstrates an advantageous performance characteristic selected from the group consisting of fluid absorption capacity, fluid absorption rate, and rewetting, wherein the advantageous performance characteristic is within at least about 80% of a similar characteristic exhibited by a crosslinked polyacrylate superabsorbent polymer, or
wherein the cumulative performance of the advantageous performance characteristics is comparable or superior to performance exhibited by the crosslinked polyacrylate superabsorbent polymer.

2. The absorbent material of claim 1, wherein the absorbent material is compostable.

3. The absorbent material of claim 1, wherein the at least one bio-based hydrogel-forming swellable polymer exhibits superabsorbent properties.

4. The absorbent material of claim 1, wherein the at least one bio-based hydrogel-forming swellable polymer is a polysaccharide.

5. The absorbent material of claim 4, wherein the polysaccharide is selected from the group consisting of dextrin, dextran, agarose, cellulose, starch, and a derivative of any of the foregoing.

6. The absorbent material of claim 4, wherein the polysaccharide is selected from the group consisting of xanthan gum, alginic acid, and sodium alginate.

7. The absorbent material of claim 1, wherein the plasticizer is selected from the group consisting of small molecules, polymeric polyols, and oligomers.

8. The absorbent material of claim 1, further comprising a surfactant.

9. The absorbent material of claim 1, further comprising one or more additional bio-based hydrogel-forming swellable polymers.

10. The absorbent material of claim 1, further comprising a second plasticizer.

11. The absorbent material of claim 10, wherein at least one of the plasticizer and the second plasticizer is a small molecule, and the small molecule is a polyol.

12. The absorbent material of claim 11, wherein the polyol is selected from the group consisting of glycerol, maltitol, and xylitol.

13. The absorbent material of claim 1, further comprising a crosslinking agent.

14. The absorbent material of claim 1, further comprising a functional additive.

15. A method of manufacturing a solid biodegradable absorbent material in a predesignated shape, wherein the solid biodegradable absorbent material demonstrates an advantageous performance characteristic selected from the group consisting of fluid absorption capacity, fluid absorption rate, and rewetting, wherein the advantageous performance characteristic is within at least about 80% of a similar characteristic exhibited by a crosslinked polyacrylate superabsorbent polymer, or wherein the cumulative performance of the advantageous performance characteristics is comparable or superior to performance exhibited by the crosslinked polyacrylate superabsorbent polymer, the method comprising:
preparing a liquid composition comprising at least one bio-based hydrogel-forming swellable polymer, a plasticizer, and a surfactant;
processing the liquid composition in a shape-forming apparatus, wherein the shape-forming apparatus is selected from the group consisting of an extruder, a mold, an electrospinner, a slot-die, and a fluid dispenser, and wherein the shape-forming apparatus forms the liquid formulation into a selected three-dimensional configuration consistent with the predesignated shape; and
solidifying the selected three-dimensional configuration, thereby producing the predesignated shape.

16. The method of claim 15, wherein:
the at least one bio-based hydrogel-forming polymer is a polysaccharide;
the plasticizer is glycerol or xylitol, and
the surfactant is capryl glucoside or hexyl glucoside.

17. The method of claim 15, wherein the predesignated shape is an elongate strand or a flattened sheet.

18. The method of claim 15, wherein the step of solidifying comprises a substep of drying the selected three-dimensional configuration to form the predesignated shape.

19. An article of manufacture comprising a disposable absorbent area, wherein the disposable absorbent area comprises the absorbent material of claim 1, and wherein the absorbent area is organized as a multilayered structure.

20. The article of manufacture of claim 19, wherein the multilayered structure comprises one or more layers of the absorbent material, and wherein the absorbent material is a foamed material.

21. The article of manufacture of claim 19, wherein the multilayered structure comprises at least one primary absorbent layer formed from the absorbent material and at least one secondary absorbent layer.

22. The article of manufacture of claim 21, wherein the at least one primary absorbent layer is formed as a sheet.

23. The article of manufacture of claim 22, wherein the sheet is penetrated with one or more apertures.

24. The article of manufacture of claim 21, wherein the at least one primary absorbent layer comprises pieces of the absorbent material overlapping each other to produce gaps that permit fluid to pass through said layer.

25. The article of manufacture of claim 21, wherein the at least one secondary absorbent layer comprises a paper-based material.

26. The article of manufacture of claim 21, wherein the at least one secondary absorbent layer is interposed between a first primary absorbent layer and a second primary absorbent layer.

27. The article of manufacture of claim 19, wherein the disposable absorbent area further comprises at least one of a specialized inner layer and a specialized outer layer.

28. The article of claim 27, wherein the specialized outer layer comprises a biopolymer having a barrier property.

29. The article of claim 27, wherein the specialized inner layer comprises a functional additive.

30. The article of manufacture of claim 19, further comprising a reusable outer shell that positions the disposable absorbent area in proximity to an anatomically advantageous area within the article of manufacture.
